(19) United States
(12) Patent Application Publication
(10) Pub. No.: US 2022/0242952 A1
(43) Pub. Date: Aug. 4, 2022

(54) ANTI-CD19 THERAPY IN PATIENTS HAVING A LIMITED NUMBER OF NK CELLS

(71) Applicant: MORPHOSYS AG, Planegg (DE)

(72) Inventors: Christian KUFFER, Planegg (DE); Guenter FINGERLE-ROWSON, Planegg (DE); Mark WINDERLICH, Planegg (DE); Jan ENDELL, Planegg (DE)

(21) Appl. No.: 17/605,285

(22) PCT Filed: May 4, 2020
(86) PCT No.: PCT/EP2020/062289

§ 371 Date: Oct. 21, 2021

(30) Foreign Application Priority Data
May 3, 2019 (EP) 19172495.4

(51) Int. Cl. C07K 16/28; A61P 35/00

(52) U.S. Cl. CPC C07K 16/2803; C07K 16/2887; A61P 35/00; C07K 2317/565; A61K 2039/507; C07K 2317/51; C07K 2317/515; C07K 2317/732; C07K 2317/56

(57) ABSTRACT

The present disclosure provides characteristics and biomarkers in patients that benefit from treatment with anti-CD19 antibodies (MOR00208, XmAb5574). Furthermore, the present application relates to anti-CD19 antibodies for the treatment of leukemia or lymphoma in patients having a peripheral NK cell count at baseline of less or equal to 100 NK cells/μl

### FIELD OF THE INVENTION

The present disclosure is directed to identifying characteristics and biomarkers in patients that benefit from treatment with anti-CD19 antibodies. Furthermore, the present disclosure relates to anti-CD19 antibodies for the treatment of leukemia or lymphoma in patients having a limited number of NK cells.

### BACKGROUND

CD19 is a 95-kDa transmembrane glycoprotein of the immunoglobulin superfamily containing two extracellular immunoglobulin-like domains and an extensive cytoplasmic tail. The protein is a pan-B lymphocyte surface receptor and is ubiquitously expressed from the earliest stages of pre-B cell development onwards until it is down-regulated during terminal differentiation into plasma cells. It is B-lymphocyte lineage specific and not expressed on hematopoietic stem cells and other immune cells, except some follicular dendritic cells. CD19 functions as a positive regulator of B cell receptor (BCR) signalling and is important for B cell activation and proliferation and in the development of humoral immune responses. It acts as a co-stimulatory molecule in conjunction with CD21 and CD81 and is critical for B cell responses to T-cell-dependent antigens. The cytoplasmic tail of CD19 is physically associated with a family of tyrosine kinases that trigger downstream signalling pathways via the src-family of protein tyrosine kinases. CD19 is an attractive target for cancers of lymphoid origin since it is highly expressed in nearly all-chronic lymphocytic leukemia (CLL) and non-Hodgkin's lymphomas (NHL), as well as many other different types of leukemias, including acute lymphocytic leukemia (ALL) and hairy cell leukemia (HCL).

MOR00208 (former name: XmAb®5574) is a humanized monoclonal antibody that targets the antigen CD19, a transmembrane protein involved in B-cell receptor signalling. MOR00208 has been engineered in the IgG Fc-region to enhance antibody-dependent cell-mediated cytotoxicity (ADCC), thus improving a key mechanism for tumor cell killing and offering potential for enhanced efficacy compared to conventional antibodies, i.e. non-enhanced antibodies. MOR00208 has or is currently being studied in several clinical trials, such as in CLL, ALL and NHL. In some of those trials, MOR00208 is used in combination with Idelalisib, Lenalidomide or Venetoclax.

In the Phase II/III trial called B-MIND, the efficacy and safety of MOR00208 in combination with Bendamustine (BEN) is evaluated in adult patients with Relapsed or Refractory Diffuse Large B-cell Lymphoma (r-r DLBCL). In this study the MOR00208 plus BEN combination is compared with the combination of Rituximab (RTX) and BEN. The chimeric mouse/human antibody Rituximab was initially approved by the U.S. Food and Drug Administration (FDA) in 1997 for treatment of patients with relapsed or refractory lowgrade or follicular CD20 positive B-cell non-Hodgkin's lymphoma (NHL). In Europe, Rituximab was approved for the treatment of NHL patients in 1998.

Recently, the number of treatment options for patients with B cell malignancies has increased and clinical efficacy of monoclonal antibodies (mAb) and mAb based therapies has been demonstrated in numerous hematologic malignancies mostly in combination with chemotherapeutics. However, a significant amount of patients with B cell malignancies is refractory or relapses after initial tumor remission in response to those combined antibody chemotherapies. Overall, variable response rates of patients to antibody therapies are observed which is based on different patient profiles. In order to further optimize success of therapy additional methods are needed to accurately predict which patients are likely to respond and/or respond best to such antibody therapies. Particular biomarkers or characteristics of patients may be found for which a particular concentration or range for each biomarker correlates with responsiveness to such therapy.

A need, therefore, exists for a method of using biomarkers or specific patient characteristics for use in connection with the treatment of cancer with therapies comprising anti-CD19 antibodies.

### SUMMARY OF INVENTION

The influence of natural killer (NK) cell count (NKCC) on the survival of patients with DLBCL treated with Rituximab, Cyclophosphamide, Doxorubicin Hydrochloride (Hydroxydaunomycin), Vincristine Sulfate (Oncovin) and Prednisone (altogether known as “R-CHOP”) was evaluated in Kim et al., Blood Research, 49:3, 162-169 (September 2014). Earlier, it was reported that peripheral NK cell count was associated with clinical outcome in patients with aalPl 2-3 DLBCL (Plonquet et al., Ann Oncol 2007; 18:1209-15).

By today, evidence has emerged which suggests that NKCC at baseline are of prognostic value for treatment of B cell lymphoma with anti-CD20 containing regimens (He et al., Blood Cancer J. 2016 Aug; 6(8); Kim et al., Blood Res. 2014 September; 49 (3):162-9; Klanova et al., Blood 2017 130:727). In large trials, involving more than 2,000 patients with previously untreated FL and DLBCL (GALLIUM, GOYA), NKCC at baseline was shown to be an independent prognostic parameter by multivariate analysis. Patients with NKCC_{high} at baseline were correlated with better prognosis compared to NKCC_{low} patients. In most studies cut-offs were selected based on the highest differential effect between both subgroups and were consistently in the range of 100 NK cells/pL blood. In general also for anti-CD19 antibody therapy a positive prognosis for NKCC_{high} patients (>100 NK cells/pL blood) was disclosed in WO2017/207574 and in the MOR00208C201 study a cut-off of at least 100 NK cells/pL was used as prognostic for the treatment outcome of MOR00208 monotherapy in DLBCL and FL.

However there is also a significant number of NKCC_{low} patients suffering hematologic cancer. Accordingly, those patients are considered to have a dismal prognosis based on their low NKCC which translates into a particular high unmet medical need for this particular patient subgroup.

The present disclosure relates to improved methods for the treatment of NKCC_{low} patients suffering B cell malignancies, such as, non-Hodgkin's lymphoma (NHL), chronic lymphocytic leukemia (CLL) and/or acute lymphoblastic leukemia (ALL). The present disclosure in particular relates to an antibody specific for CD19 for the treatment of B cell malignancies, such as, non-Hodgkin's lymphoma, chronic lymphocytic leukemia and/or acute lymphoblastic leukemia in NKCC_{low} patients.

In the present disclosure the ADCC activity of MOR00208 was compared to the ADCC activity of the anti-CD20 antibody Rituximab in B-cell tumor cell lines at various effector to target (E:T) ratios. Rituximab can be described as being the gold standard treatment in those indications. Target cell lines were derived from DLBCL, MCL and CLL with CD19 and CD20 levels, which are in the range of expression levels on B-cell tumor patient samples as reported by Boltez̆ar et al. 2018, Ginaldi et al. 1998 and Olejniczak et al. 2006. Respective E:T ratios in ADCC assays on several cell lines were used to elucidate a potential correlation between superiority of MOR00208 vs Rituximab and NKCC. The obtained data showed increasing relative benefit of MOR00208 with decreasing E:T ratios, thus providing a rationale for MOR00208 superiority in the NKCC_{low} subgroup. Based on available data for cut-off determination the NKCC_{low} subgroup is defined as patients having less or equal to 100 NK cells/μl at baseline.

Therefore, patients diagnosed with a B-cell malignancy, such as, non-Hodgkin's lymphoma, chronic lymphocytic leukemia and/or acute lymphoblastic leukemia and having a baseline peripheral NK cell count at baseline of less or equal to 100 cells/μl are more likely to benefit from MOR00208 treatment in comparison to available therapy.

The present disclosure provides an anti-CD19 antibody for use in the treatment of hematological cancer patients wherein said patients have a peripheral NK cell count at baseline of less or equal to 100 cells/μl. In one embodiment said patient is resistant, non-responsive or inadequately responsive to treatment by one and not more than three prior lines of therapy, including one anti-CD20 targeting therapy (e.g. the antibody rituximab). In a further embodiment said patient is not eligible for high-dose chemotherapy and autologous stem cell transplantation. In a preferred embodiment said patient is a human.

In an embodiment the anti-CD19 antibody for use in the treatment of hematological cancer patients comprises an HCDR1 region comprising the sequence SYVMH (SEQ ID NO: 1), an HCDR2 region comprising the sequence NPYNDG (SEQ ID NO: 2), an HCDR3 region comprising the sequence GTYYYGTRVFDY (SEQ ID NO: 3), an LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6).

In a further embodiment the anti-CD19 antibody for use in the treatment of hematological cancer patients comprises a variable heavy chain of the sequence

(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS

and a variable light chain of the sequence

(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

In another embodiment of the present disclosure the anti-CD19 antibody is a human, humanized or chimeric antibody. In another embodiment of the present disclosure the anti-CD19 antibody is of the IgG isotype. In another embodiment the antibody is IgG1, IgG2 or IgG1/IgG2 chimeric. In another embodiment of the present disclosure the isotype of the anti-CD19 antibody is engineered to enhance antibody-dependent cell-mediated cytotoxicity. In another embodiment the heavy chain constant region of the anti-CD19 antibody comprises amino acids 239D and 332E, wherein the Fc numbering is according to the EU index as in Kabat. In another embodiment the antibody is IgG1, IgG2 or IgG1/IgG2 and the chimeric heavy chain constant region of the anti-CD19 antibody comprises amino acids 239D and 332E, wherein the Fc numbering is according to the EU index as in Kabat.

In a further embodiment the anti-CD19 antibody for use in the treatment of hematological cancer patients comprises a heavy chain having the sequence

(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTIPP
MLDSDGSFFLYSKLTVDKSRWQQGNVFSCSVMHEALHNHYTQKSLSLSPG
K

and a light chain having the sequence

(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

### BRIEF DESCRIPTION OF THE DRAWINGS

FIG. 1: Representative ADCC assays of MOR00208 and Rituximab at increasing E:T ratios. Results for specific killing expressed as % dead target cell mediated by MOR00208 (black) or Rituximab (white) and NK cells from healthy donors are depicted for three target cell lines MEC-1, JVM-2 and Toledo. Exemplary data obtained with NK cells from one representative donor in a single experiment are shown. Bars and error bars represent geometric mean and geometric standard deviation of specific killing measured in triplicates from an individual experiment.

FIG. 2: Representative ADCC assays of MOR00208 and Rituximab at increasing E:T ratios. The corresponding ratios of specific killing normalized to Rituximab mediated by MOR00208 (blue) or Rituximab (orange) and NK cells from healthy donors are depicted for three target cell lines MEC-1, JVM-2 and Toledo. The scatter plots illustrate the individual values for the specific killing ratio of MOR00208 (triangle) and Rituximab (circle) versus the median Rituximab value of a representative experiment. The dotted lines represent the geometric mean with its 95% bootstrap confidence interval.

FIG. 3: Specific killing ratios of MOR00208 and Rituximab with NK cells isolated from 33 healthy donors and MEC-1, JVM-2 and Toledo cells in ADCC assays at increasing E:T ratios. ADCC activity of MOR00208 (white triangle) and Rituximab (black circle) was analysed with MEC-1 cells (8 NK cell donors, 2 independent experiments; 9 NK cell donors in single experiments), JVM-2 (8 NK cell donors, 2 independent experiments per donor) and Toledo cells (10 NK cell donors, 2 independent experiments). The ratio of specific killing was calculated from the % specific killing determined for each antibody by normalizing to the median value of Rituximab. Respective circles or triangles represent the geometric mean value of either one or two independent experiments performed in triplicates with NK cells from one individual blood donor.

### DETAILED DESCRIPTION OF THE INVENTION

Non-Hodgkin's lymphoma (“NHL”) is a heterogeneous malignancy originating from lymphocytes. In the United States (U.S.), the incidence is estimated at 65,000/year with mortality of approximately 20,000 (American Cancer Society, 2006; and SEER Cancer Statistics Review). The disease can occur in all ages, the usual onset begins in adults over 40 years, with the incidence increasing with age. NHL is characterized by a clonal proliferation of lymphocytes that accumulate in the lymph nodes, blood, bone marrow and spleen, although any major organ may be involved. The current classification system used by pathologists and clinicians is the World Health Organization (WHO) Classification of Tumours, which organizes NHL into precursor and mature B-cell or T-cell neoplasms. The PDQ is currently dividing NHL as indolent or aggressive for entry into clinical trials. The indolent NHL group is comprised primarily of follicular subtypes, small lymphocytic lymphoma, MALT (mucosa-associated lymphoid tissue), and marginal zone; indolent encompasses approximately 50% of newly diagnosed B-cell NHL patients. Aggressive NHL includes patients with histologic diagnoses of primarily diffuse large B cell (DLBL, “DLBCL”, or DLCL) (40% of all newly diagnosed patients have diffuse large cell), Burkitt's, and mantle cell (“MCL”). The clinical course of NHL is highly variable. A major determinant of clinical course is the histologic subtype. Most indolent types of NHL are considered to be incurable disease. Patients respond initially to either chemotherapy or antibody therapy and most will relapse. Studies to date have not demonstrated an improvement in survival with early intervention. In asymptomatic patients, it is acceptable to “watch and wait” until the patient becomes symptomatic or the disease pace appears to be accelerating. Over time, the disease may transform to a more aggressive histology. The median survival is 8 to 10 years, and indolent patients often receive 3 or more treatments during the treatment phase of their disease. Initial treatment of the symptomatic indolent NHL patient historically has been combination chemotherapy. The most commonly used agents include: cyclophosphamide, vincristine and prednisone (CVP); or cyclophosphamide, adriamycin, vincristine, prednisone (CHOP). Approximately 70% to 80% of patients will respond to their initial chemotherapy, duration of remissions last on the order of 2-3 years. Ultimately the majority of patients relapse. The discovery and clinical use of the anti-CD20 antibody, rituximab, has provided significant improvements in response and survival rate. The current standard of care for most patients is rituximab +CHOP (R-CHOP) or rituximab +CVP (R-CVP). Rituximab therapy has been shown to be efficacious in several types of NHL, and is currently approved as a first line treatment for both indolent (follicular lymphoma) and aggressive NHL (diffuse large B cell lymphoma). However, there are significant limitations of anti-CD20 monoclonal antibody (mAb), including primary resistance (50% response in relapsed indolent patients), acquired resistance (50% response rate upon re-treatment), rare complete response (2% complete resonse rate in relapsed population), and a continued pattern of relapse. Finally, many B cells do not express CD20, and thus many B-cell disorders are not treatable using anti-CD20 antibody therapy.

In addition to NHL there are several types of leukemias that result from dysregulation of B cells. Chronic lymphocytic leukemia (also known as “chronic lymphoid leukemia” or “CLL”), is a type of adult leukemia caused by an abnormal accumulation of B lymphocytes. In CLL, the malignant lymphocytes may look normal and mature, but they are not able to cope effectively with infection. CLL is the most common form of leukemia in adults. Men are twice as likely to develop CLL as women. However, the key risk factor is age. Over 75% of new cases are diagnosed in patients over age 50. More than 10,000 cases are diagnosed every year and the mortality is almost 5,000 a year (American Cancer Society, 2006; and SEER Cancer Statistics Review). CLL is an incurable disease but progresses slowly in most cases. Many people with CLL lead normal and active lives for many years. Because of its slow onset, early-stage CLL is generally not treated since it is believed that early CLL intervention does not improve survival time or quality of life. Instead, the condition is monitored over time. Initial CLL treatments vary depending on the exact diagnosis and the progression of the disease. There are dozens of agents used for CLL therapy. Combination chemotherapy regimens such as FCR (fludarabine, cyclophosphamide and rituximab), and BR (Ibrutinib and rituximab) are effective in both newly-diagnosed and relapsed CLL. Allogeneic bone marrow (stem cell) transplantation is rarely used as a first-line treatment for CLL due to its risk.

Another type of leukemia is Small lymphocytic lymphoma (“SLL”) that is considered a CLL variant that lacks the clonal lymphocytosis required for the CLL diagnosis, but otherwise shares pathological and immunophenotypic features (Campo et al., 2011). The definition of SLL requires the presence of lymphadenopathy and/or splenomegaly. Moreover, the number of B lymphocytes in the peripheral blood should not exceed 5×109/L. In SLL, the diagnosis should be confirmed by histopathologic evaluation of a lymph node biopsy whenever possible (Hallek et al., 2008). The incidence of SLL is approximately 25% of CLL in the US (Dores et al., 2007).

Another type of leukemia is acute lymphoblastic leukemia (ALL), also known as acute lymphocytic leukemia. ALL is characterized by the overproduction and continuous multiplication of malignant and immature white blood cells (also known as lymphoblasts) in the bone marrow. ‘Acute’ refers to the undifferentiated, immature state of the circulating lymphocytes (“blasts”), and that the disease progresses rapidly with life expectancy of weeks to months if left untreated. ALL is most common in childhood with a peak incidence of 4-5 years of age. Children of age 12-16 die more easily from it than others. Currently, at least 80% of childhood ALL are considered curable. Under 4,000 cases are diagnosed every year and the mortality is almost 1,500 a year (American Cancer Society, 2006; and SEER Cancer Statistics Review).

The use of a CD19 antibody in non-specific B cell lymphomas is discussed in WO2007076950 (US2007154473), which are both incorporated by reference. The use of a CD19 antibody in CLL, NHL and ALL is described in Scheuermann et al., CD19 Antigen in Leukemia and Lymphoma Diagnosis and Immunotherapy, Leukemia and Lymphoma, Vol. 18, 385-397 (1995), which is incorporated by reference in its entirety.

Additional antibodies specific for CD19 are described in WO2005012493 (U.S. Pat. No. 7,109,304), WO2010053716 (U.S. Ser. No. 12/266,999) (Immunomedics); WO2007002223 (U.S. Pat. No. 8,097,703) (Medarex); WO2008022152 (U.S. Ser. No. 12/377,251) and WO2008150494 (Xencor), WO2008031056 (U.S. Ser. No. 11/852,106) (Medimmune); WO 2007076950 (U.S. Ser. No. 11/648,505) (Merck Patent GmbH); WO 2009/052431 (U.S. Ser. No. 12/253,895) (Seattle Genetics); and WO2010095031 (U.S. Ser. No. 12/710,442) (Glenmark Pharmaceuticals), WO2012010562 and WO2012010561 (International Drug Development), WO2011147834 (Roche Glycart), and WO 2012/156455 (Sanofi), which are all incorporated by reference in their entireties.

A pharmaceutical composition includes an active agent, e.g. an antibody for therapeutic use in humans. A pharmaceutical composition may additionally include pharmaceutically acceptable carriers or excipients.

### Definitions

The term “CD19” refers to the protein known as CD19, having the following synonyms: B4, B-lymphocyte antigen CD19, B-lymphocyte surface antigen B4, CVID3, Differentiation antigen CD19, MGC12802, and T-cell surface antigen Leu-12.

Human CD19 has the amino acid sequence of:

(SEQ ID NO: 13)
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPG
PPSEKAWQPGWTVNVEGSGELFRWNVSDLGGLGCGLKNRSSEGPSSPSGK
LMSPKLYVWAKDRPEIWEGEPPCLPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKRKRMTDPTRRFFKVTPPPGSGPQNQYGN
VLSLPTPTSGLGRAQRWAAGLGGTAPSYGNPSSDVQADGALGSRSPPGVG
PEEEEGEGYEEPDSEEDSEFYENDSNLGQDQLSQDGSGYENPEDEPLGPE
DEDSFSNAESYENEDEELTQPVARTMDFLSPHGSAWDPSREATSLGSQSY
EDMRGILYAAPQLRSIRGQPGPNHEEDADSYENMDNPDGPDPAWGGGGRM
GTWSTR

“MOR00208” is an anti-CD19 antibody. The amino acid sequences are provided in Table 1. “MOR00208” and “XmAb 5574” are used as synonyms to describe the antibody shown in Table 1. The MOR00208 antibody is described in U.S. patent application Ser. No. 12/377,251, which is incorporated by reference in its entirety. U.S. patent application Ser. No. 12/377,251 describes the antibody named 4G7 H1.52 Hybrid S239D/I332E/4G7 L1.155 (later named MOR00208).

The term “antibody” as used herein refers to a protein comprising at least two heavy (H) chains and two light (L) chains inter-connected by disulfide bonds, which interacts with an antigen. Each heavy chain is comprised of a variable heavy chain region (abbreviated herein as VH) and a heavy chain constant region. The heavy chain constant region is comprised of three domains, CH1, CH2 and CH3. Each light chain is comprised of a variable light chain region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FR's arranged from amino-terminus to carboxy-terminus in the following order: FR1, CDR1, FR2, CDR2, FR3, CDR3, and FR4. The variable regions of the heavy and light chains contain a binding domain that interacts with an antigen. The term “antibody” includes for example, monoclonal antibodies, human antibodies, humanized antibodies, camelised antibodies and chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology.

The phrase “antibody fragment”, as used herein, refers to one or more portions of an antibody that retain the ability to specifically interact with (e.g., by binding, steric hindrance, stabilizing spatial distribution) an antigen. Examples of binding fragments include, but are not limited to, a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1126-1136). Antibody fragments can be grafted into scaffolds based on polypeptides such as Fibronectin type III (Fn3) (see U.S. Pat. No. 6,703,199, which describes fibronectin polypeptide monobodies). Antibody fragments can be incorporated into single chain molecules comprising a pair of tandem Fv segments (VH-CH1-VH-CH1) which, together with complementary light chain polypeptides, form a pair of antigen-binding sites (Zapata et al., (1995) Protein Eng. 8:1057-1062; and U.S. Pat. No. 5,641,870).

“Administered” or “administration” includes but is not limited to delivery of a drug by an injectable form, such as, for example, an intravenous, intramuscular, intradermal or subcutaneous route or mucosal route, for example, as a nasal spray or aerosol for inhalation or as an ingestible solution, capsule or tablet. Preferably, the administration is by an injectable form.

The term “effector function” refers to those biological activities attributable to the Fc region of an antibody, which vary with the antibody isotype. Non-limiting examples of antibody effector functions include C1q binding and complement dependent cytotoxicity (CDC); Fc receptor binding and antibody-dependent cell-mediated cytotoxicity (ADCC) and/or antibody-dependent cellular phagocytosis (ADCP); down regulation of cell surface receptors (e.g. B cell receptor); and B cell activation.

“Antibody-dependent cell-mediated cytotoxicity” or “ADCC” refers to a form of cytotoxicity in which antibodies bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express FcyRlll only, whereas monocytes express FcγRI, FcγRII, and FcγRIII.

“Complement-dependent cytotoxicity” or “CDC” refers to the lysis of a target cell in the presence of complement. Activation of the classical complement pathway is initiated by the binding of the first component of the complement system (C1q) to antibodies (of the appropriate subclass) of the present disclosure, which are bound to their cognate antigen.

“Antibody-dependent cellular phagocytosis” or “ADCP” refers to a mechanism of elimination of antibody-coated target cells by internalization by phagocytic cells, such as macrophages or dendritic cells.

The term “hematologic cancer” includes blood-borne tumors and diseases or disorders involving abnormal cell growth and/or proliferation in tissues of hematopoietic origin, such as lymphomas, leukemias, and myelomas.

“Subject” or “patient” as used in this context refers to any mammal, including rodents, such as mouse or rat, and primates, such as cynomolgus monkey (Macaca fascicularis), rhesus monkey (Macaca mulatta) or humans (Homo sapiens). Preferably, the subject or patient is a primate, most preferably a human.

The terms “engineered” or “modified” as used herein includes manipulation of nucleic acids or polypeptides by synthetic means (e.g. by recombinant techniques, in vitro peptide synthesis, by enzymatic or chemical coupling of peptides or some combination of these techniques). Preferably, the antibodies or antibody fragments according to the present disclosure are engineered or modified to improve one or more properties, such as antigen binding, stability, half-life, effector function, immunogenicity, safety and the like. Preferably the antibodies or antibody fragments according to the present disclosure are engineered or modified to improve effector function, such as ADCC.

“Variant” as used herein refers to a polypeptide that differs from a reference polypeptide by one or more modifications for example amino acid substitutions, insertions or deletions.

The term “antagonistic” antibody as used herein refers to an antibody or antibody fragment that interacts with an antigen and partially or fully inhibits or neutralizes a biological activity or function or any other phenotypic characteristic of a target antigen.

The “Fc region” is used to define the C-terminal region of an immunoglobulin heavy chain. The Fc region of an immunoglobulin generally comprises two constant domains, a CH2 domain and a CH3 domain. Unless otherwise specified herein, numbering of amino acid residues in the Fc region is according to the EU numbering system, also called the EU index, as described in Kabat et al., Sequences of Proteins of Immunological Interest, 5th Ed. Public Health Service, National Institutes of Health, Bethesda, Md., 1991.

The antibody which is administered according to the present disclosure is administered to the patient in a therapeutically effective amount. A “therapeutically effective amount” refers to an amount sufficient to provide some improvement of the clinical manifestations of a given disease or disorder. The amount that is effective for a particular therapeutic purpose will depend on the severity of the disease or injury as well as on the weight and general state of the subject. It will be understood that determination of an appropriate dosage may be achieved, using routine experimentation, by constructing a matrix of values and testing different points in the matrix, all of which is within the ordinary skills of a trained physician or clinical scientist.

“Baseline” or “at baseline” means prior to administration of the desired therapy. For example, prior to administration of the desired anti-CD19 antibody.

A receiver operating characteristic (ROC) analysis can be used to analyze the predictivity, sensitivity, specificity to determine the cut-offs for potential biomarkers, such as NK cell counts. The following additional methods exist for estimating an optimal cut-off: a) “Max.Accuracy”—the cut-off which maximize the accuracy; b) “Max.DOR”—the cut-off which maximize the diagnostic odds ratio; c) “Error.rate”—the cut-off which minimizes the error rate; d) “Max.Accuracy.area”—the cut-off which maximize the accuracy area; e) “Max.Sens+Spec”—the cut-off which maximize the sum of sensitivity with specificity; f) “Max.Youden”—the cut-off which maximize the Youden index; g) “Se=Sp”—the cut-off which Sensitivity is equal to Specificity; h) “Min.ROC.Dist”—the cut-off which minimize the distance between the curve and the upper left corner of the graph; i) “Max.Efficiency”—the cut-off which maximize the efficiency; and j) “Min.MCT”—the cut-off which minimize the misclassification cost term. See Lopez-Raton, M., Rodriguez-Alvarez, M.X, Cadarso-Suarez, C. and Gude-Sampedro, F. (2014). Optimal Cutpoints: An R Package for Selecting Optimal Cutpoints in Diagnostic Tests. Journal of Statistical Software 61 (8), 1-36.

Antibodies specific to CD19 have also been tested preclinically in combination with other drugs. For example, MOR00208 had been tested in combination with nitrogen mustards, purine analogs, thalidomide analogs, phosphoinositide 3-kinase inhibitor, BCL-2 inhibitors and bruton's tyrosine kinase (BTK) inhibitors.

“In combination” refers to the administration of one therapy in addition to another therapy. As such, “in combination with” includes simultaneous (e.g., concurrent) and consecutive administration in any order. By way of non-limiting example, a first therapy (e.g., agent, such as an anti-CD19 antibody) may be administered before (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., pharmaceutical agent or a pharmaceutically acceptable salt thereof) to a patient. In some embodiments, the term “combination” means that the anti-CD19 antibody and the pharmaceutical agent or a pharmaceutically acceptable salt thereof are administered simultaneously or consecutivley. In certain embodiments, the anti-CD19 antibody and the pharmaceutical agent or a pharmaceutically acceptable salt thereof are administered in separate compositions, i.e., wherein the anti-CD19 antibody and the pharmaceutical agent or a pharmaceutically acceptable salt thereof are administered each in a separate unit dosage form. It is understood that the anti-CD19 antibody and the pharmaceutical agent or a pharmaceutically acceptable salt thereof are administered on the same day or on different days and in any order as according to an appropriate dosing protocol.

A “nitrogen mustard” is a nonspecific DNA alkylating agent used as chemotherapy. Alkylating agents add an alkyl group (CnH2n+1) to nucleic acid bases, e.g., adding an alkyl group to the guanine base of DNA at the number 7 nitrogen atom of the imidazole ring. The alkylation steps result in the formation of interstrand cross-links (ICLs). These ICLs are highly cytotoxic, since they block fundamental metabolic processes such as replication and transcription. Nitrogen mustards include cyclophosphamide, chlorambucil, uramustine, ifosfamide, melphalan and bendamustine.

Bendamustine is marketed under the names Ribomustin®,and Treanda®, and is also known as SDX-105. Bendamustine is for the treatment of chronic lymphocytic leukemias (CLL), indolent B-cell non-Hodgkin's lymphoma (NHL), and other lymphomas. Bendamustine has the following structure:

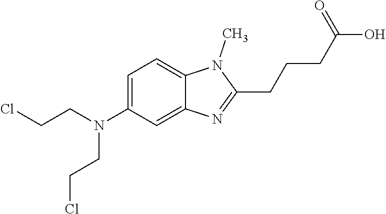 [SMILES: Cn1c(CCCC(=O)O)nc2cc(N(CCCl)CCCl)ccc21]

A purine analog is an antimetabolite, which mimics the structure of metabolic purines, thereby interfering with the synthesis of nucleic acids. Fludarabine, for example, may be incorporated into RNA and DNA by substituting for the purine nucleotides, adenine and guanine. Purine analogs inhibit growth of fast proliferating cells of an individual, e.g. cancer cells, bone marrow cells or cells present in the gastrointestinal tract. Purine analogs include mercaptopurine, azathioprine, thioguanine and fludarabine. Fludarabine or fludarabine phosphate (Fludara®) is a chemotherapy drug used in the treatment of chronic lymphocytic leukemia and indolent non-Hodgkins lymphomas. Fludarabine is a purine analog. Fludarabine inhibits DNA synthesis by interfering with ribonucleotide reductase and DNA polymerase and is S phase-specific (since these enzymes are highly active during DNA replication). Fludarabine has the following structure:

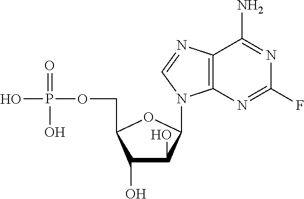

A “thalidomide analog” includes, but is not limited to, thalidomide itself, lenalidomide (CC-5013, Revlimid™), Pomalidomide (CC4047, Actimid™) and the compounds disclosed in WO2002068414 and WO2005016326, which are incorporated by reference in their entireties. The term refers to a synthetic chemical compound using the thalidomide structure as a backbone (e.g., side groups have been added or such groups have been deleted from the parent structure). The analog differs in structure from thalidomide and its metabolite compounds such as by a difference in the length of an alkyl chain, a molecular fragment, by one or more functional groups, or 4w5wwwwa change in ionization. The term “thalidomide analog” also includes the metabolites of thalidomide. Thalidomide analogs include the racemic mixture of the S- and the R-enantiomer of a respective compound and the S-enantiomer or to the R-enantiomer individually. The racemic mixture is preferred.

Thalidomide analogs include compounds such as lenalidomide which has the following structure:

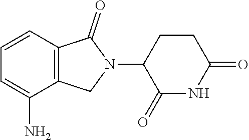 [SMILES: Nc1cccc2c1CN(C1CCC(=O)NC1=O)C2=O]

A “phosphoinositide 3-kinase inhibitor” is a class of medical drug that functions by inhibiting one or more of the phosphoinositide 3-kinase enzymes, which are part of the PI3K/AKT/mTOR pathway, an important signalling pathway for many cellular functions such as growth control, metabolism and translation initiation.

There are a number of different classes and isoforms of PI3Ks. Class 1 PI3Ks have a catalytic subunit known as p110, with four types (isoforms)—p110 alpha, p110 beta, p110 gamma and p110 delta. Current inhibitors being studied inhibit one or more isoforms of the class I PI3Ks.

Phosphoinositide 3-kinase inhibitors include at least Idelalisib, Duvelisib and Copanlisib. Idelalisib is marketed by Gilead Sciences, Inc. (trade name Zydelig, also named GS-1101 or CAL-101). Idelalisib is is currently labelled for the treatment of relapsed chronic lymphocytic leukemia (CLL), in combination with rituximab, in patients for whom rituximab alone would be considered appropriate therapy due to other co-morbidities; relapsed follicular B-cell non-Hodgkin lymphoma (FL) in patients who have received at least two prior systemic therapies; relapsed small lymphocytic lymphoma (SLL) in patients who have received at least two prior systemic therapies. The substance acts as a phosphoinositide 3-kinase inhibitor; more specifically, it blocks P1106, the delta isoform of the enzyme phosphoinositide 3-kinase.

The formula of Idelalisib is:

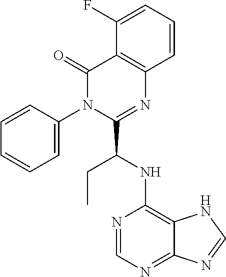 [SMILES: CC[C@H](Nc1ncnc2nc[nH]c12)c1nc2cccc(F)c2c(=O)n1-c1ccccc1]

A “Bruton's tyrosine kinase (BTK) inhibitor” is a class of drug that functions by inhibiting the tyrosine-protein kinase BTK enzyme, which plays an important role in B-cell development. Specifically, BTK contains a PH domain that binds phosphatidylinositol (3,4,5)-trisphosphate (PIP3). PIP3 binding induces Btk to phosphorylate phospholipase C, which in turn hydrolyzes PIP2, a phosphatidylinositol, into two second messengers, inositol triphosphate (IP3) and diacylglycerol (DAG), which then go on to modulate the activity of downstream proteins during B-cell signalling.

Bruton's tyrosine kinase (BTK) inhibitors include Ibrutinib. Ibrutinib is marketed by Pharmacyclics, Inc and Johnson & Johnson's Janssen Pharmaceutical (trade name Imbruvica, also named PCI-32765). Ibrutinib is currently labelled for the treatment of patients with Mantle cell lymphoma (MCL) who have received at least one prior therapy, Chronic lymphocytic leukemia (CLL) who have received at least one prior therapy, Chronic lymphocytic leukemia with 17p deletion, and Waldenstrom's macroglobulinemia. The formula of Ibrutinib is 1-[(3R)-3-[4-amino-3-(4-phenoxyphenyl)-1H-pyrazolo[3,4-d]pyrimidin-1-yl]-1-piperidinyl]-2-propen-1-one and has the following structure:

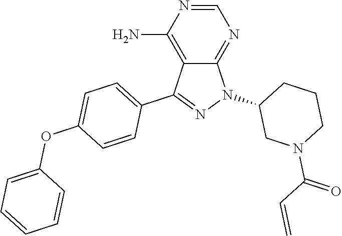 [SMILES: C=CC(=O)N1CCC[C@@H](n2nc(-c3ccc(Oc4ccccc4)cc3)c3c(N)ncnc32)C1]

A “BCL-2 inhibitor” is a class of drug that functions by inhibiting anti-apoptotic B-cell lymphoma-2 (Bcl-2) protein, leading to programmed cell death of cells. BCL-2 inhibitor include venetoclax. Venetoclax is marketed by Abbvie and Genentech (trade name VENCLEXTA™, also known as GDC-0199, ABT-199, and RG7601). Venetoclax is currently labelled for the treatment of patients with chronic lymphocytic leukemia (CLL) with 17p deletion, as detected by an FDA approved test, who have received at least one prior therapy. “Venetoclax is described in U.S. Pat. Nos. 8,546,399 and 9,174,982, which are all incorporated by reference in their entireties. The formula of venetoclax is

4-(4-{[2-(4-Chlorophenyl)-4,4-dimethyl-1-cyclohexen-1-yl]nethyl}-1-piperazinyl)-N-({3-nitro-4-[(tetrahydro-2H-pyran-4-ylmethyl)amino]phenyl}sulfonyl)-2-(1H-pyrrolo[2,3-b]pyridin-5-yloxy) benzamide and has the following structure:

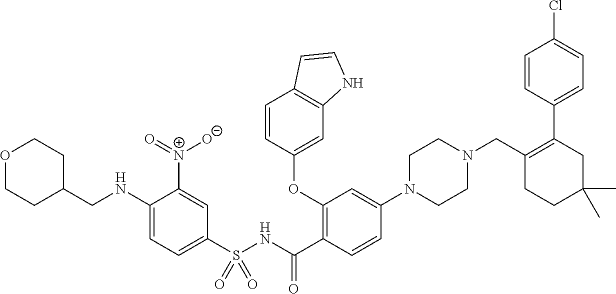 [SMILES: CC1(C)CCC(CN2CCN(c3ccc(C(=O)NS(=O)(=O)c4ccc(NCC5CCOCC5)c([N+](=O)[O-])c4)c(Oc4ccc5cc[nH]c5c4)c3)CC2)=C(c2ccc(Cl)cc2)C1]

### Embodiments

In other embodiments the present disclosure refers to an anti-CD19 antibody for use in the treatment of hematological cancer patients wherein said patients have a peripheral NK cell count at baseline of less or equal to 100 cells/μl, less or equal to 90 cells/μl, less or equal to 80 cells/μl, less or equal to 70 cells/μl, less or equal to 60 cells/μl or less or equal to 50 cells/μl.

In other embodiments the present disclosure refers to an anti-CD19 antibody for use in the treatment of hematological cancer patients wherein said patients have a peripheral NK cell count at baseline of less than 100 cells/μI, less than 90 cells/μl, less than 80 cells/μl, less than 70 cells/μl, less than 60 cells/μl or less than 50 cells/μl.

In other embodiments the present disclosure refers to an anti-CD19 antibody for use in the treatment of hematological cancer patients wherein said patients have a peripheral NK cell count at baseline at baseline of 1 to maximum 100 cells/μl, of 10 to maximum 100 cells/μl, of 20 to maximum 100 cells/μl, of 30 to maximum 100 cells/μl, of 40 to maximum 100 cells/μl, of 50 to maximum 100 cells/μl, of 60 to maximum 100 cells/μl, of 70 to maximum 100 cells/μl, or of 80 to maximum 100 cells/μl.

In other embodiments the present disclosure refers to the use of an anti-CD19 antibody for use in the treatment of hematological cancer patients wherein said patients have a peripheral NK cell count at baseline of less than 100 cells/μl, less than 90 cells/μl, less than 80 cells/μl, less than 70 cells/μl, less than 60 cells/μl or less than 50 cells/μl.

In other embodiments the present disclosure refers to the use of an an anti-CD19 antibody for the treatment of hematological cancer patients wherein said patients have a peripheral NK cell count at baseline of maximum 100 cells/μl, of maximum 90 cells/μl, of maximum 80 cells/μl, of maximum 70 cells/μl, of maximum 60 cells/μl, of maximum 50 cells/μl.

In other embodiments the present disclosure refers to the use of an an anti-CD19 antibody for the treatment of hematological cancer patients wherein said patients have a peripheral NK cell count at baseline of 1 to maximum 100 cells/μl, of 10 to maximum 100 cells/μl, of 20 to maximum 100 cells/μl, of 30 to maximum 100 cells/μl, of 40 to maximum 100 cells/μl, of 50 to maximum 100 cells/μl, of 60 to maximum 100 cells/μl, of 70 to maximum 100 cells/μl, or of 80 to maximum 100 cells/μl.

In an embodiment the anti-CD19 antibody for use in the treatment of hematological cancer patients comprises a variable heavy chain of the sequence

(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS

and a variable light chain of the sequence

(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK

or a variable heavy chain and and a variable light chain that has at least 80%, at least 90%, at least 95%, at least 96%, at least 97%, at least 98% or at least 99% identity to the variable heavy chain of SEQ ID NO: 7 and to the variable light chain of SEQ ID NO: 8.

In an embodiment the anti-CD19 antibody for use in the treatment of hematological cancer patients comprises a variable heavy chain of the sequence

(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS

and a variable light chain of the sequence

(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK

or a variable heavy chain and and a variable light chain that has at least 80%, at least 90%, at least 95%, at least 96%, at least 97%, at least 98% or at least 99% identity to the variable heavy chain of SEQ ID NO: 7 and to the variable light chain of SEQ ID NO: 8, wherein the anti-CD19 antibody comprises an HCDR1 region comprising the sequence SYVMH (SEQ ID NO: 1), an HCDR2 region comprising the sequence NPYNDG (SEQ ID NO: 2), an HCDR3 region comprising the sequence GTYYYGTRVFDY (SEQ ID NO: 3), an LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6). In another embodiment the heavy chain region of the anti-CD19 antibody comprises amino acids 239D and 332E, wherein the Fc numbering is according to the EU index as in Kabat.

In a further embodiment the anti-CD19 antibody for the treatment of hematological cancer patients comprises a heavy chain having the sequence

(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLOSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNOVSLTCLVKGFYPSDIAVEWESNGQPENNYKTIPP
MLDSDGSFFLYSKLTVDKSRWQQGNVFSCSVMHEALHNHYTQKSLSLSPG
K

and a light chain having the sequence

(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

or a heavy chain and and a light chain that has at least 80%, at least 90%, at least 95%, at least 96%, at least 97%, at least 98% or at least 99% identity to the heavy chain of SEQ ID NO: 7 and to the light chain of SEQ ID NO: 8.

In a further embodiment the anti-CD19 antibody for the treatment of hematological cancer patients comprises a heavy chain having the sequence

(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLOSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNOVSLTCLVKGFYPSDIAVEWESNGQPENNYKTIPP
MLDSDGSFFLYSKLTVDKSRWQQGNVFSCSVMHEALHNHYTQKSLSLSPG
K

and a light chain having the sequence

(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

or a heavy chain and and a light chain that has at least 80%, at least 90%, at least 95%, at least 96%, at least 97%, at least 98% or at least 99% identity to the heavy chain of SEQ ID NO: 7 and to the light chain of SEQ ID NO: 8 and wherein the anti-CD19 antibody comprises an HCDR1 region comprising the sequence SYVMH (SEQ ID NO: 1), an HCDR2 region comprising the sequence NPYNDG (SEQ ID NO: 2), an HCDR3 region comprising the sequence GTYYYGTRVFDY (SEQ ID NO: 3), an LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6). In another embodiment the heavy chain region of the anti-CD19 antibody comprises amino acids 239D and 332E, wherein the Fc numbering is according to the EU index as in Kabat.

In other embodiments the present disclosure refers to an anti-CD19 antibody for the treatment of hematological cancer patients wherein said patients have a peripheral NK cell count at baseline of less or equal to 100 cells/μl, less or equal to 90 cells/μl, less or equal to 80 cells/μl, less or equal to 70 cells/μl, less or equal to 60 cells/μl or less or equal to 50 cells/μl. In one embodiment of the present disclosure said hematological cancer patients after said treatment have
(i) a progression-free survival (PFS) of at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 24 months, at least 30 months, at least 36 months, at least 42 months, at least 48 months or at least 54 months;
(ii) an objective response rate (ORR) of at least 30%, at least 40%, at least 50%, at least 60%, at least 70%, at least 80% or at least 80%;
(iii) a duration of response (DoR) over at least at least 10 months, at least 12 months, at at least 14 months, at least 16 months, at least 18 months, at least 20 months, at least 24 months, at least 30 months, at least 36 months, at least 42 months, at least 48 months or at least 54 months;
(iv) an overall survival (OS) of at least at least 10 months, at least 12 months, at at least 14 months, at least 16 months, at least 18 months, at least 20 months, at least 24 months, at least 30 months, at least 36 months, at least 42 months, at least 48 months or at least 54 months or
(v) a combination of one or more of the foregoing. In another embodiment of the present disclosure said anti-CD19 antibody is administered in combination with a pharmaceutical agent as disclosed herein.

In other embodiments the present disclosure refers to an anti-CD19 antibody for the treatment of hematological cancer patients wherein said patients have a peripheral NK cell count at baseline of less than 100 cells/μl, less than 90 cells/μl, less than 80 cells/μl, less than 70 cells/μl, less than 60 cells/μl or less than 50 cells/μl. In one embodiment of the present disclosure said hematological cancer patients after said treatment have
(i) a progression-free survival (PFS) of at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 24 months, at least 30 months, at least 36 months, at least 42 months, at least 48 months or at least 54 months;
(ii) an objective response rate (ORR) of at least 30%, at least 40%, at least 50%, at least 60%, at least 70%, at least 80% or at least 80%;
(iii) a duration of response (DoR) over at least at least 10 months, at least 12 months, at at least 14 months, at least 16 months, at least 18 months, at least 20 months, at least 24 months, at least 30 months, at least 36 months, at least 42 months, at least 48 months or at least 54 months;
(iv) an overall survival (OS) of at least at least 10 months, at least 12 months, at at least 14 months, at least 16 months, at least 18 months, at least 20 months, at least 24 months, at least 30 months, at least 36 months, at least 42 months, at least 48 months or at least 54 months or
(v) a combination of one or more of the foregoing. In another embodiment of the present disclosure said anti-CD19 antibody is administered in combination with a pharmaceutical agent as disclosed herein.

In other embodiments the present disclosure refers to an anti-CD19 antibody for the treatment of hematological cancer patients wherein said patients have a peripheral NK cell count at baseline of less or equal to 100 cells/μl, less or equal to 90 cells/μl, less or equal to 80 cells/μl, less or equal to 70 cells/μl, less or equal to 60 cells/μl or less or equal to 50 cells/μl and wherein said anti-CD19 antibody increases one or more of the following features:
(i) the progression-free survival (PFS),
(ii) the objective response rate (ORR),
(iii) the duration of response (DoR),
(iv) the overall survival (OS),
(v) the time to progression (TTP).

In another embodiments said one or more of the features (i) to (v) are increased relative to the treatment comprising an anti-CD20 antibody. In a further embodiment said one or more of the features (i) to (v) are increased in comparison to the treatment comprising an anti-CD20 antibody and a chemotherapeutic. In a further embodiment said anti-CD20 antibody is rituximab or a biosimilar thereof. In further embodiments said one or more of the features (i) to (v) are increased in comparison to the treatment comprising an anti-CD20 antibody and one or more of cyclophosphamide, adriamycin, vincristine or prednisone. In a further embodiment said one or more of the features (i) to (v) are increased in comparison to the treatment comprising R-CHOP.

In other embodiments the present disclosure refers to an anti-CD19 antibody for the treatment of hematological cancer patients wherein said patients have a peripheral NK cell count at baseline of less than 100 cells/μl, less than 90 cells/μl, less than 80 cells/μl, less than 70 cells/μl, less than 60 cells/μl or less than 50 cells/μl and wherein the administration of said anti-CD19 antibody results in improved progression-free survival (PFS), improved objective response rate (ORR), improved duration of response (DoR), improved overall survival (OS) or improved time to progression (TTP).

In other embodiments the present disclosure refers to an anti-CD19 antibody for the treatment of hematological cancer patients wherein said patients have a peripheral NK cell count at baseline of less or equal to 100 cells/μl, less or equal to 90 cells/μl, less or equal to 80 cells/μl, less or equal to 70 cells/μl, less or equal to 60 cells/μl or less or equal to 50 cells/μl and wherein the administration of said anti-CD19 antibody results in improved progression-free survival (PFS) relative to the administration of an anti-CD20 antibody, improved objective response rate (ORR) relative to the administration of an anti-CD20 antibody, improved duration of response (DoR) relative to the administration of an anti-CD20 antibody, improved overall survival (OS) relative to the administration of an anti-CD20 antibody or improved time to progression (TTP) relative to the administration of an anti-CD20 antibody.

In other embodiments the present disclosure refers to an anti-CD19 antibody for the treatment of hematological cancer patients wherein said patients have a peripheral NK cell count at baseline of less or equal to 100 cells/μl, less or equal to 90 cells/μl, less or equal to 80 cells/μl, less or equal to 70 cells/μl, less or equal to 60 cells/μl or less or equal to 50 cells/μl and wherein the administration of said anti-CD19 antibody results in improved progression-free survival (PFS) relative to the administration of an anti-CD20 antibody and a chemotherapeutic, improved objective response rate (ORR) relative to the administration of an anti-CD20 antibody and a chemotherapeutic, improved duration of response (DoR) relative to the administration of an anti-CD20 antibody and a chemotherapeutic, improved overall survival (OS) relative to the administration of an anti-CD20 antibody and a chemotherapeutic or improved time to progression (TTP) relative to the administration of an anti-CD20 antibody and a chemotherapeutic. In a further embodiment said anti-CD20 antibody is rituximab or a biosimilar thereof. In further embodiments said chemotherapeutic comprises one or more of cyclophosphamide, adriamycin, vincristine or prednisone.

In other embodiments the present disclosure refers to an anti-CD19 antibody for the treatment of hematological cancer patients wherein said patients have a peripheral NK cell count at baseline of less or equal to 100 cells/μl, less or equal to 90 cells/μl, less or equal to 80 cells/μl, less or equal to 70 cells/μl, less or equal to 60 cells/μl or less or equal to 50 cells/μl and wherein the administration of said anti-CD19 antibody results in improved progression-free survival (PFS) relative to the administration of R-CHOP, improved objective response rate (ORR) relative to the administration of R-CHOP, improved duration of response (DoR) relative to the administration of R-CHOP, improved overall survival (OS) relative to the administration of R-CHOP or improved time to progression (TTP) relative to the administration of R-CHOP.

In one embodiment the present disclosure provides an anti-CD19 antibody wherein said anti-CD19 antibody is administered in a concentration of 12 mg/kg.

In a further embodiment, the anti-CD19 antibody is administered weekly, bi-weekly or monthly. In a further embodiment the anti-CD19 antibody is administered weekly for the first 3 months and bi-weekly for at least the next 3 months. In a further embodiment, the anti-CD19 antibody is administered weekly for the first 3 months. In a further embodiment the anti-CD19 antibody is administered weekly for the first 3 months and bi-weekly for at least the next 3 months. In another embodiment the anti-CD19 antibody is administered weekly for the first 3 months, bi-weekly for the next 3 months and monthly thereafter. In yet another embodiment the anti-CD19 antibody is administered weekly for the first 3 months, bi-weekly for the next 3 months and monthly thereafter.

### Combinations

The present disclosure provides an anti-CD19 antibody for the treatment of hematological cancer patients wherein said patients have a peripheral NK cell count at baseline of less than 100 cells/ μl and wherein said anti-CD19 antibody is administered in combination with one or more pharmaceutical agents. In one embodiment of the present disclosure said anti-CD19 antibody is administered in combination with a pharmaceutical agent. In another embodiment of the present disclosure said anti-CD19 antibody is administered in combination with one or more additional pharmaceutical agents or an additional pharmaceutical agent. In one aspect said pharmaceutical agent is an additional pharmaceutical agent.

In one embodiment of the present disclosure said pharmaceutical agent is a biologic or a chemotherapeutic agent. In another embodiment of the present disclosure said pharmaceutical agent is a therapeutic antibody or antibody fragment, a nitrogen mustard, a purine analog, a thalidomide analog, a phosphoinositide 3-kinase inhibitor, a BCL-2 inhibitor or a bruton's tyrosine kinase (BTK) inhibitor. In a further embodiment said pharmaceutical agent is rituximab, R-CHOP, cyclophosphamide, chlorambucil, uramustine, ifosfamide, melphalan, bendamustine, mercaptopurine, azathioprine, thioguanine, fludarabine, thalidomide, lenalidomide, pomalidomide, idelalisib, duvelisib, copanlisib, ibrutinib or venetoclax.

In another embodiment the present disclosure provides an anti-CD19 antibody for use in the treatment of hematological cancer patients wherein said patients have a peripheral NK cell count at baseline of less or equal to 100 cells/μl and wherein said anti-CD19 antibody is administered in combination with rituximab, R-CHOP, cyclophosphamide, chlorambucil, uramustine, ifosfamide, melphalan, bendamustine, mercaptopurine, azathioprine, thioguanine, fludarabine, thalidomide, lenalidomide, pomalidomide, idelalisib, duvelisib, copanlisib, ibrutinib or venetoclax. In a further embodiment the present disclosure provides an anti-CD19 antibody for the treatment of hematological cancer patients wherein said patients have a peripheral NK cell count at baseline of less or equal to 100 cells/μl and wherein said anti-CD19 antibody is administered in combination with bendamustine.

### Indications and Patients

The present disclosure provides an anti-CD19 antibody for the treatment of hematological cancer patients wherein said patients have a peripheral NK cell count at baseline of less or equal to 100 cells/μl and wherein said hematologic cancer patient has chronic lymphocytic leukemia (CLL), non-Hodgkin's lymphoma (NHL), small lymphocytic lymphoma (SLL) or acute lymphoblastic leukemia (ALL). In another embodiment said hematologic cancer patient has non-Hodgkin's lymphoma. In further embodiments the non-Hodgkin's lymphoma is selected from the group consisting of follicular lymphoma, small lymphocytic lymphoma, mucosa-associated lymphoid tissue, marginal zone lymphoma, diffuse large B cell lymphoma, Burkitt's lymphoma and mantle cell lymphoma. In further embodiments the non-Hodgkin's lymphoma is Relapsed or Refractory Diffuse Large B-cell Lymphoma (r-r DLBCL). In another embodiment said hematologic cancer patient has diffuse large B cell lymphoma and is not eligible for High-Dose Chemotherapy (HDC) and/or Autologous Stem-Cell Transplantation (ASCT). In another embodiment said hematologic cancer patient has Relapsed or Refractory Diffuse Large B-cell Lymphoma (r-r DLBCL) and is not eligible for High-Dose Chemotherapy (HDC) and/or Autologous Stem-Cell Transplantation (ASCT).

In another embodiment said hematologic cancer patient has diffuse large B cell lymphoma wherein the patient is selected based on one or more of the following criteria:
1. Age 18 years
2. Histologically confirmed diagnosis, according to the World Health Organization (WHO, 2008) classification, of: DLBCL NOS, THRLBCL, EBV-positive DLBCL, composite lymphoma with a DLBCL component with a DLBCL relapse subsequent to DLBCL treatment, disease transformed from an earlier diagnosis of low grade lymphoma (i.e. an indolent pathology such as follicular lymphoma, marginal zone lymphoma) into DLBCL with a DLBCL relapse subsequent to DLBCL treatment.
3. Fresh tumour tissue for central pathology review must be provided as an adjunct to participation in this study. Should it not be possible to obtain a fresh tumour tissue sample, archival paraffin embedded tumour tissue acquired ≤3 years prior to screening for this protocol must be available for this purpose.
4. Patients must have:
   1. relapsed or refractory DLBCL
   2. at least one bidimensionally measurable disease site. The lesion must have a greatest transverse diameter of ≥1.5 cm and greatest perpendicular diameter of ≥1.0 cm at baseline. The lesion must be positive on PET scan
   3. received at least one, but no more than three previous systemic therapy lines for the treatment of DLBCL. At least one previous therapy line must have included a CD20-targeted.
   4. ECOG 0 to 2
5. Patients after failure of ASCT or patients considered in the opinion of the investigator currently not eligible for HDC with subsequent ASCT.
6. Patients must meet the following laboratory criteria at Screening:
   a) ANC≥1.5×109/L (unless secondary to bone marrow involvement by DLBCL)
   b) PLTs≥90×109/L (unless secondary to bone marrow involvement by DLBCL) and absence of active bleeding
   c) total serum bilirubin≤2.5×ULN unless secondary to Gilbert's syndrome (or pattern consistent with Gilbert's) or documented liver involvement by lymphoma. Patients with Gilbert's syndrome or documented liver involvement by lymphoma may be included if their total bilirubin is ≤5×ULN
   d) ALT, AST and AP≤3×ULN or <5×ULN in cases of documented liver involvement by lymphoma
   e) serum creatinine≤2.0×ULN or creatinine clearance must be mL/min calculated using a standard Cockcroft-Gault formula (Cockroft & Gault, 1976)
7. For a female of childbearing potential (FCBP), a negative pregnancy test must be confirmed before enrolment. An FCBP must commit to take highly effective contraceptive precautions without interruption during the study and for 3, 6 or 12 months after the last dose of MOR00208, BEN or RTX respectively, whichever is later. An FCBP must refrain from breastfeeding and donating blood or oocytes during the course of the study and for 3, 6 or 12 months after the last dose of MOR00208, BEN or RTX respectively, whichever is later. Restrictions concerning blood donations apply as well to females who are not of childbearing potential.
8. Males must use an effective barrier method of contraception without interruption during the study and for 3, 6 or 12 months after the last dose of MOR00208, BEN or RTX respectively, whichever is later, if the patient is sexually active with an FCBP. Males must refrain from donating blood or sperm during study participation and for 3, 6 or 12 months after the last dose of MOR00208, BEN or RTX respectively, whichever is later.
9. In the opinion of the investigator, the patients must:
   a) be able to comply with all study-related procedures, medication use, and evaluations
   b) be able to understand and give informed consent
   c) not be considered to be potentially unreliable and/or not cooperative.

In another embodiment said hematologic cancer patient has diffuse large B cell lymphoma wherein the patient is excluded based on one or more of the following exclusion criteria:
1. Patients who have: any other histological type of lymphoma including, e.g., primary mediastinal (thymic) large B-cell lymphoma (PMBL) or Burkitt's lymphoma, primary refractory DLBCL, patients with known “double/triple hit” DLBCL genetics, CNS lymphoma involvement in present or past medical history
2. Patients who had a major surgery less than 30 days prior to Day 1 dosing
3. Patients who have, within 14 days prior to Day 1 dosing:
   a) not discontinued CD20-targeted therapy, chemotherapy, radiotherapy, investigational anticancer therapy or other lymphoma-specific therapy
   b) received live vaccines
   c) required parenteral antimicrobial therapy for active, intercurrent systemic infections
4. Patients who:
   a) in the opinion of the investigator, have not recovered sufficiently from the adverse toxic effects of prior therapies, major surgeries or significant traumatic injuries
   b) were previously treated with CD19-targeted therapy or BEN
   c) have a history of previous severe allergic reactions to compounds of similar biological or chemical composition to MOR00208, RTX, murine proteins or BEN, or the excipients contained in the study drug formulations
   d) have undergone ASCT within a period of ≤3 months prior to signing the informed consent form. Patients who have a more distant history of ASCT must exhibit full haematological recovery before enrolment into the study.
   e) have undergone previous allogeneic stem cell transplantation
   f) concurrently use other anticancer or experimental treatments
5. Prior history of malignancies other than DLBCL, unless the patient has been free of the disease for ≥3 years prior to Screening. Exceptions to the 3-year time limit include history of the following:
   a) basal cell carcinoma of the skin
   b) squamous cell carcinoma of the skin
   c) carcinoma in situ of the cervix, breast and bladder
   d) incidental histological finding of prostate cancer (Tumour/Node/Metastasis [TNM] stage of T1a or T1b)
6. Patients with:
   a) positive hepatitis B and/or C serology
   b) known seropositivity for or history of active viral infection with HIV
   c) evidence of active, severe uncontrolled systemic infections or sepsis
   d) a history or evidence of severely immunocompromised state
   e) a history or evidence of severe hepatic impairment (total serum bilirubin >3 mg/dL), jaundice unless secondary to Gilbert's syndrome or documented liver involvement by lymphoma
   f) a history or evidence of clinically significant cardiovascular, cerebrovascular,

CNS and/or other disease that, in the investigator's opinion, would preclude participation in the study or compromise the patient's ability to give informed consent

### Method of treatment

The present disclosure provides a method of treating hematological cancer patients by the administration of an anti-CD19 antibody wherein said patients have a peripheral NK cell count at baseline of less or equal to 100 cells/μl, less or equal to 90 cells/μl, less or equal to 80 cells/μl, less or equal to 70 cells/μl, less or equal to 60 cells/μl or less or equal to 50 cells/μl.

The present disclosure provides a method of treating hematological cancer patients by the administration of an anti-CD19 antibody wherein said patients have a peripheral NK cell count at baseline of less than 100 cells/μl, less than 90 cells/μl, less than 80 cells/μl, less than 70 cells/μl, less than 60 cells/μl or less than 50 cells/μl.

In another embodiment the present disclosure refers to pharmaceutical compositions comprising an anti-CD19 antibody as disclosed herein for the use in the treatment of hematological cancer. In another embodiment the present disclosure refers to the use of said pharmaceutical compositions comprising an anti-CD19 antibody as disclosed herein in the preparation of a medicament for the treatment of hematological cancer. In another embodiment the present disclosure refers to the use of said pharmaceutical composition comprising an anti-CD19 antibody as disclosed herein for the treatment of hematological cancer. In another embodiment said hematological cancer is chronic lymphocytic leukemia (CLL), non-Hodgkin's lymphoma (NHL), small lymphocytic lymphoma (SLL) or acute lymphoblastic leukemia (ALL). In another embodiment said hematologic cancer is non-Hodgkin's lymphoma. In further embodiments the non-Hodgkin's lymphoma is selected from the group consisting of follicular lymphoma, small lymphocytic lymphoma, mucosa-associated lymphoid tissue, marginal zone lymphoma, diffuse large B cell lymphoma, Burkitt's lymphoma and mantle cell lymphoma. In further embodiments the non-Hodgkin's lymphoma is Relapsed or Refractory Diffuse Large B-cell Lymphoma (r-r DLBCL). In another embodiment said hematologic cancer patient has diffuse large B cell lymphoma and is not eligible for High-Dose Chemotherapy (HDC) and/or Autologous Stem-Cell Transplantation (ASCT).

In another aspect, provided herein is a method of treating hematological cancer in a patient, wherein said patient has a peripheral NK cell count at baseline of less or equal to 100 cells/μl, less or equal to 90 cells/μl, less or equal to 80 cells/μl, less or equal to 70 cells/μl, less or equal to 60 cells/μl or less or equal to 50 cells/μl, the method comprising administering a pharmaceutical composition comprising a therapeutically effective amount of the anti-CD19 antibody as disclosed herein. In one embodiment said patient is resistant, non-responsive or inadequately responsive to treatment by one and not more than three prior lines of therapy, including one anti-CD20 targeting therapy (e.g. the antibody rituximab). In a further embodiment said patient is not be eligible for high-dose chemotherapy and autologous stem cell transplantation. In a preferred embodiment said patient is a human. In alternative aspects said patient is a rodent, such as a rat or a mouse. In another embodiment said patient suffers a hematologic cancer such non-Hodgkin's lymphoma. In further embodiments the non-Hodgkin's lymphoma is selected from the group consisting of follicular lymphoma, small lymphocytic lymphoma, mucosa-associated lymphoid tissue, marginal zone lymphoma, diffuse large B cell lymphoma, Burkitt's lymphoma and mantle cell lymphoma. In further embodiments the non-Hodgkin's lymphoma is Relapsed or Refractory Diffuse Large B-cell Lymphoma (r-r DLBCL).

In another aspect the present disclosure provides the use of an anti-CD19 antibody in the manufacture of a medicament for use in the treatment of a hematological cancer patient wherein said patient has a peripheral NK cell count at baseline of less or equal to 100 cells/μl, less or equal to 90 cells/μl, less or equal to 80 cells/μl, less or equal to 70 cells/μl, less or equal to 60 cells/μl or less or equal to 50 cells/μl.

In another aspect the present disclosure provides the use of an anti-CD19 antibody in the manufacture of a medicament for use in the treatment of hematological cancer. In another embodiment the present disclosure refers to the use of said anti-CD19 antibody as disclosed herein in the preparation of a medicament for the treatment of hematological cancer. In another embodiment the present disclosure refers to the use of said pharmaceutical composition comprising an anti-CD19 antibody as disclosed herein for the treatment of hematological cancer. In another embodiment said hematological cancer is chronic lymphocytic leukemia (CLL), non-Hodgkin's lymphoma (NHL), small lymphocytic lymphoma (SLL) or acute lymphoblastic leukemia (ALL). In another embodiment said hematologic cancer is non-Hodgkin's lymphoma. In further embodiments the non-Hodgkin's lymphoma is selected from the group consisting of follicular lymphoma, small lymphocytic lymphoma, mucosa-associated lymphoid tissue, marginal zone lymphoma, diffuse large B cell lymphoma, Burkitt's lymphoma and mantle cell lymphoma. In further embodiments the non-Hodgkin's lymphoma is Relapsed or Refractory Diffuse Large B-cell Lymphoma (r-r DLBCL). In another embodiment said hematologic cancer patient has diffuse large B cell lymphoma and is not eligible for High-Dose Chemotherapy (HDC) and/or Autologous Stem-Cell Transplantation (ASCT).

In some embodiments, the anti-CD19 antibody as disclosed herein is administered intravenously. In other aspects the anti-CD19 antibody as disclosed herein is administered subcutaneously, intra-articularly or intra-spinally.

### Method

The present disclosure provides a method of selecting a hematological cancer patient who is expected to benefit from the therapeutic administration of an anti-CD19 antibody, said method comprising the following steps:
a) providing a blood sample obtained from said patient prior to treatment with said anti-CD19 antibody,
b) determining the peripheral NK cell count, and
c) selecting the patient on the basis of the patient has a peripheral NK cell count at baseline of less or equal to 100 cells/ μl.

In an embodiment of the present disclosure the method further comprises the following step:
d) treatment of the selected patient with an anti-CD19 antibody.

The present disclosure provides a method of selecting a hematological cancer patient who is expected to benefit from the therapeutic administration of an anti-CD19 antibody in combination with a pharmaceutical agent, said method comprising the following steps:
a) providing a blood sample obtained from said patient prior to treatment with said anti-CD19 antibody,
b) determining the peripheral NK cell count, and
c) selecting the patient on the basis of the patient has a peripheral NK cell count at baseline of less or equal to 100 cells/ μl.

In an embodiment of the present disclosure the method further comprises the following step:
d) treatment of the selected patient with an anti-CD19 antibody.

The present disclosure provides a method of selecting a hematological cancer patient who is predicted to benefit from the therapeutic administration of an anti-CD19 antibody, said method comprising the following steps:
a) providing a blood sample obtained from said patient prior to treatment with said anti-CD19 antibody,
b) determining the peripheral NK cell count, and
c) selecting the patient on the basis of the patient has a peripheral NK cell count at baseline of less or equal to 100 cells/μl.

In an embodiment of the present disclosure the method further comprises the following step:
d) treatment of the selected patient with an anti-CD19 antibody.

The present disclosure provides a method of selecting a hematological cancer patient who is predicted to benefit from the therapeutic administration of an anti-CD19 antibody in combination with a pharmaceutical agent, said method comprising the following steps:
a) providing a blood sample obtained from said patient prior to treatment with said anti-CD19 antibody,
b) determining the peripheral NK cell count, and
c) selecting the patient on the basis of the patient has a peripheral NK cell count at baseline of less or equal to 100 cells/μl.

In an embodiment of the present disclosure the method further comprises the following step:
d) treatment of the selected patient with an anti-CD19 antibody.

In one embodiment of the present disclosure said pharmaceutical agent administered in combination with said anti-CD19 antibody is a biologic or a chemotherapeutic agent. In another embodiment of the present disclosure said pharmaceutical agent is a therapeutic antibody or antibody fragment, a nitrogen mustard, a purine analog, a thalidomide analog, a phosphoinositide 3-kinase inhibitor, a BCL-2 inhibitor or a bruton's tyrosine kinase (BTK) inhibitor. In a further embodiment said pharmaceutical agent is rituximab, R-CHOP, cyclophosphamide, chlorambucil, uramustine, ifosfamide, melphalan, bendamustine, mercaptopurine, azathioprine, thioguanine, fludarabine, thalidomide, lenalidomide, pomalidomide, idelalisib, duvelisib, copanlisib, ibrutinib or venetoclax.

The present disclosure provides a method of identifying a hematological cancer patient who is predicted to benefit from the therapeutic administration of an anti-CD19 antibody, said method comprising the following steps:
a) providing a blood sample obtained from said patient prior to treatment with said anti-CD19 antibody,
b) determining the peripheral NK cell count, and
c) selecting the patient on the basis of the patient has a peripheral NK cell count at baseline of less or equal to 100 cells/μl.

In an embodiment of the present disclosure the method further comprises the following step:
d) treatment of the selected patient with an anti-CD19 antibody.

The present disclosure provides a method of treating hematological cancer by the administration of an anti-CD19 antibody to a hematological cancer patient wherein said patient was selected according to a method comprising the following steps:
a) providing a blood sample obtained from said patient prior to treatment with said anti-CD19 antibody,
b) determining the peripheral NK cell count, and
c) selecting the patient on the basis of the patient has a peripheral NK cell count at baseline of less or equal to 100 cells/μl.

In an embodiment of the present disclosure the method further comprises the following step:
d) treatment of the selected patient with an anti-CD19 antibody.

The present disclosure provides a method of selecting a hematological cancer patient who is predicted to benefit from the therapeutic administration of an anti-CD19 antibody, said method comprising the following steps:
a) providing a blood sample obtained from said patient prior to treatment with said anti-CD19 antibody,
b) determining the peripheral NK cell count, and
c) selecting the patient on the basis of the patient has a peripheral NK cell count at baseline of less or equal to 100 cells/μl.

In an embodiment of the present disclosure the method further comprises the following step:
d) treatment of the selected patient with an anti-CD19 antibody.

In another embodiment of the present disclosure the predicted benefit from the therapeutic administration of an anti-CD19 antibody is improved progression-free survival (PFS), improved objective response rate (ORR), improved duration of response (DoR), improved overall survival (OS) or improved time to progression (TTP) or a combination thereof.

In another embodiment of the present disclosure the predicted benefit from the therapeutic administration of an anti-CD19 antibody is improved progression-free survival (PFS) relative to the administration of an anti-CD20 antibody, improved objective response rate (ORR) relative to the administration of an anti-CD20 antibody, improved duration of response (DoR) relative to the administration of an anti-CD20 antibody, improved overall survival (OS) relative to the administration of an anti-CD20 antibody or improved time to progression (TTP) relative to the administration of an anti-CD20 antibody or a combination thereof.

In another embodiment of the present disclosure the predicted benefit from the therapeutic administration of an anti-CD19 antibody is
(i) a progression-free survival (PFS) of at least 8 months, at least 9 months, at least 10 months, at least 11 months, at least 12 months, at least 13 months, at least 14 months, at least 15 months, at least 16 months, at least 17 months, at least 18 months, at least 19 months or at least 20 months;
(ii) an objective response rate (ORR) of at least 30%, at least 40%, at least 50%, at least 60%, at least 70%, at least 80% or at least 80%;
(iii) a duration of response (DoR) over at least at least 10 months, at least 12 months, at at least 14 months, at least 16 months, at least 18 months, at least 20 months, at least 24 months, at least 30 months, at least 36 months, at least 42 months, at least 48 months or at least 54 months;
(iv) an overall survival (OS) of at least at least 10 months, at least 12 months, at at least 14 months, at least 16 months, at least 18 months, at least 20 months, at least 24 months, at least 30 months, at least 36 months, at least 42 months, at least 48 months or at least 54 months or
(v) a combination of one or more of the foregoing. In another embodiment of the present disclosure said anti-CD19 antibody is administered in combination with a pharmaceutical agent as disclosed herein.

In another embodiment of the present disclosure the predicted benefit from the therapeutic administration of an anti-CD19 antibody is improved progression-free survival (PFS) relative to the administration of an anti-CD20 antibody and a chemotherapeutic, improved objective response rate (ORR) relative to the administration of an anti-CD20 antibody and a chemotherapeutic, improved duration of response (DoR) relative to the administration of an anti-CD20 antibody and a chemotherapeutic, improved overall survival (OS) relative to the administration of an anti-CD20 antibody and a chemotherapeutic or improved time to progression (TTP) relative to the administration of an anti-CD20 antibody and a chemotherapeutic. In a further embodiment said anti-CD20 antibody is rituximab or a biosimilar thereof. In further embodiments said chemotherapeutic comprises one or more of cyclophosphamide, adriamycin, vincristine or prednisone.

In another embodiment of the present disclosure the predicted benefit from the therapeutic administration of an anti-CD19 antibody is improved progression-free survival (PFS) relative to the administration of R-CHOP, improved objective response rate (ORR) relative to the administration of R-CHOP, improved duration of response (DoR) relative to the administration of R-CHOP, improved overall survival (OS) relative to the administration of R-CHOP or improved time to progression (TTP) relative to the administration of R-CHOP.

In another embodiment of the present disclosure said predicted benefit from the therapeutic administration of an anti-CD19 antibody is an increase of one or more of the following features:
(i) the progression-free survival (PFS),
(ii) the objective response rate (ORR),
(iii) the duration of response (DoR),
(iv) the overall survival (OS),
(v) the time to progression (TTP).

In another embodiment said increase of one or more of the features (i) to (v) are in comparison to the treatment comprising an anti-CD20 antibody. In a further embodiment said increase of one or more of the features (i) to (v) are in comparison to the treatment comprising an anti-CD20 antibody and a chemotherapeutic. In a further embodiment said anti-CD20 antibody is rituximab or a biosimilar thereof. In a further embodiment said increase of one or more of the features (i) to (v) are in comparison to the treatment comprising an anti-CD20 antibody and one or more of cyclophosphamide, adriamycin, vincristine or prednisone. In a further embodiment said increase of one or more of the features (i) to (v) are in comparison to the treatment comprising R-CHOP.

In an embodiment of the present disclosure said hematologic cancer patient of said method of selecting a hematological cancer patient who is predicted to benefit from the therapeutic administration of an anti-CD19 antibody has chronic lymphocytic leukemia (CLL), non-Hodgkin's lymphoma (NHL), small lymphocytic lymphoma (SLL) or acute lymphoblastic leukemia (ALL). In a further embodiment said hematologic cancer patient has non-Hodgkin's lymphoma. In a further embodiment said hematologic cancer patient has non-Hodgkin's lymphoma, wherein the non-Hodgkin's lymphoma is selected from the group consisting of follicular lymphoma, small lymphocytic lymphoma, mucosa-associated lymphoid tissue, marginal zone lymphoma, diffuse large B cell lymphoma, Burkitt's lymphoma and mantle cell lymphoma. In a further embodiment said hematologic cancer patient has Relapsed or Refractory Diffuse Large B-cell Lymphoma (r-r DLBCL).

In a further embodiment of the present disclosure the anti-CD1 9 antibody of the method of selecting a hematological cancer patient who is predicted to benefit from the therapeutic administration of an anti-CD19 antibody comprises an HCDR1 region comprising the sequence SYVMH (SEQ ID NO: 1), an HCDR2 region comprising the sequence NPYNDG (SEQ ID NO: 2), an HCDR3 region comprising the sequence GTYYYGTRVFDY (SEQ ID NO: 3), an LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6). In another embodiment said anti-CD19 antibody comprises a variable heavy chain of the sequence

(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS

and a variable light chain of the sequence

(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

In a further embodiment said anti-CD19 antibody comprises a heavy chain having the sequence

(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLOSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNOVSLTCLVKGFYPSDIAVEWESNGQPENNYKTIPP
MLDSDGSFFLYSKLTVDKSRWQQGNVFSCSVMHEALHNHYTQKSLSLSPG
K

and a light chain having the sequence

(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

In another embodiment the anti-CD19 antibody for the treatment of hematological cancer patients comprises a variable heavy chain and and a variable light chain that has at least 80%, at least 90%, at least 95%, at least 96%, at least 97%, at least 98% or at least 99% identity to the variable heavy chain of SEQ ID NO: 7 and to the variable light chain of SEQ ID NO: 8.

In an embodiment the anti-CD19 antibody for the treatment of hematological cancer patients comprises a variable heavy chain and and a variable light chain that has at least 80%, at least 90%, at least 95%, at least 96%, at least 97%, at least 98% or at least 99% identity to the variable heavy chain of SEQ ID NO: 7 and to the variable light chain of SEQ ID NO: 8, wherein the anti-CD19 antibody comprises an HCDR1 region comprising the sequence SYVMH (SEQ ID NO: 1), an HCDR2 region comprising the sequence NPYNDG (SEQ ID NO: 2), an HCDR3 region comprising the sequence GTYYYGTRVFDY (SEQ ID NO: 3), an LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6). In another embodiment the heavy chain region of the anti-CD19 antibody comprises amino acids 239D and 332E, wherein the Fc numbering is according to the EU index as in Kabat.

In a further embodiment the anti-CD19 antibody for the treatment of hematological cancer patients comprises a heavy chain and and a light chain that has at least 80%, at least 90%, at least 95%, at least 96%, at least 97%, at least 98% or at least 99% identity to the heavy chain of SEQ ID NO: 7 and to the light chain of SEQ ID NO: 8.

In a further embodiment the anti-CD19 antibody for the treatment of hematological cancer patients comprises a heavy chain and and a light chain that has at least 80%, at least 90%, at least 95%, at least 96%, at least 97%, at least 98% or at least 99% identity to the heavy chain of SEQ ID NO: 7 and to the light chain of SEQ ID NO: 8 and wherein the anti-CD19 antibody comprises an HCDR1 region comprising the sequence SYVMH (SEQ ID NO: 1), an HCDR2 region comprising the sequence NPYNDG (SEQ ID NO: 2), an HCDR3 region comprising the sequence GTYYYGTRVFDY (SEQ ID NO: 3), an LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6). In another embodiment the heavy chain region of the anti-CD19 antibody comprises amino acids 239D and 332E, wherein the Fc numbering is according to the EU index as in Kabat.

In a further embodiment the present disclosure provides a kit comprising means to determine the peripheral NK cell count of a hematological cancer patient to be treated with an anti-CD19 antibody.

In a further embodiment the present disclosure refers to the use of the peripheral NK cell count as a biomarker to predict the susceptibility of a hematological cancer patient to the treatment with an anti-CD19 antibody. In another embodiments the peripheral NK cell count at baseline is less or equal to 100 cells/μl, less or equal to 90 cells/μl, less or equal to 80 cells/μl, less or equal to 70 cells/μl, less or equal to 60 cells/μl or less or equal to 50 cells/μl. In other embodiments the peripheral NK cell count at baseline is 1 to maximum 100 cells/μl, 10 to maximum 100 cells/μl, 20 to maximum 100 cells/μl, 30 to maximum 100 cells/μl, 40 to maximum 100 cells/μl, 50 to maximum 100 cells/μl, 60 to maximum 100 cells/μl, 70 to maximum 100 cells/μl, or 80 to maximum 100 cells/μl.

In a further embodiment the present disclosure refers to the use of the peripheral NK cell count as a biomarker to predict the susceptibility of a hematological cancer patient to the treatment with an anti-CD19 antibody. In another embodiments the peripheral NK cell count at baseline is less than 100 cells/μl, less than 90 cells/μl, less than 80 cells/μl, less than 70 cells/μl, less than 60 cells/μl or less than 50 cells/μl. In other embodiments the peripheral NK cell count at baseline is 1 to maximum 100 cells/μl, 10 to maximum 100 cells/μl, 20 to maximum 100 cells/μl, 30 to maximum 100 cells/μl, 40 to maximum 100 cells/μl, 50 to maximum 100 cells/μl, 60 to maximum 100 cells/μl, 70 to maximum 100 cells/μl, or 80 to maximum 100 cells/μl.

### Antibody sequences

TABLE 1

| | SEQ ID NO: | Amino Acids |
|---|---|---|
| HCDR1 | SEQ ID NO: 1 | SYVMH |
| HCDR2 | SEQ ID NO: 2 | NPYNDG |
| HCDR3 | SEQ ID NO: 3 | GTYYYGTRVFDY |
| LCDR1 | SEQ ID NO: 4 | RSSKSLQNVNGNTYLY |
| LCDR2 | SEQ ID NO: 5 | RMSNLNS |
| LCDR3 | SEQ ID NO: 6 | MQHLEYPIT |
| VH | SEQ ID NO: 7 | EVQLVESGGGLVKPGGSLKLSCAASGYTFTSY |
| | | VMHWVRQAPGKGLEWIGYINPYNDGTKYNEK |
| | | FQGRVTISSDKSISTAYMELSSLRSEDTAMYYC |
| | | ARGTYYYGTRVFDYWGQGTLVTVSS |
| VL | SEQ ID NO: 8 | DIVMTQSPATLSLSPGERATLSCRSSKSLQNV |
| | | NGNTYLYWFQQKPGQSPQLLIYRMSNLNSGV |
| | | PDRFSGSGSGTEFTLTISSLEPEDFAVYYCMQ |
| | | HLEYPITFGAGTKLEIK |
| Heavy chain constant | SEQ ID NO: 9 | ASTKGPSVFPLAPSSKSTSGGTAALGCLVKDY |
| domain | | FPEPVTVSWNSGALTSGVHTFPAVLQSSGLYS |
| | | LSSVVTVPSSSLGTQTYICNVNHKPSNTKVDK |
| | | KVEPKSCDKTHTCPPCPAPELLGGPDVFLFPP |
| | | KPKDTLMISRTPEVTCVVVDVSHEDPEVQFNW |
| | | YVDGVEVHNAKTKPREEQFNSTFRVVSVLTVV |
| | | HQDWLNGKEYKCKVSNKALPAPEEKTISKTKG |
| | | QPREPQVYTLPPSREEMTKNQVSLTCLVKGFY |
| | | PSDIAVEWESNGQPENNYKTTPPMLDSDGSF |
| | | FLYSKLTVDKSRWQQGNVFSCSVMHEALHNH |
| | | YTQKSLSLSPGK |
| Light chain constant | SEQ ID NO: 10 | RTVAAPSVFIFPPSDEQLKSGTASVVCLLNNFY |
| domain | | PREAKVQWKVDNALQSGNSQESVTEQDSKD |
| | | STYSLSSTLTLSKADYEKHKVYACEVTHQGLS |
| | | SPVTKSFNRGEC |
| Full Heavy chain | SEQ ID NO: 11 | EVQLVESGGGLVKPGGSLKLSCAASGYTFTSY |
| | | VMHWVRQAPGKGLEWIGYINPYNDGTKYNEK |
| | | FQGRVTISSDKSISTAYMELSSLRSEDTAMYYC |
| | | ARGTYYYGTRVFDYWGQGTLVTVSSASTKGP |
| | | SVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKS |
| | | CDKTHTCPPCPAPELLGGPDVFLFPPKPKDTL |
| | | MISRTPEVTCVVVDVSHEDPEVQFNWYVDGV |
| | | EVHNAKTKPREEQFNSTFRVVSVLTVVHQDW |
| | | LNGKEYKCKVSNKALPAPEEKTISKTKGQPRE |
| | | PQVYTLPPSREEMTKNQVSLTCLVKGFYPSDI |
| | | AVEWESNGQPENNYKTTPPMLDSDGSFFLYS |
| | | KLTVDKSRWQQGNVFSCSVMHEALHNHYTQK |
| | | SLSLSPGK |
| Full Light chain | SEQ ID NO: 12 | DIVMTQSPATLSLSPGERATLSCRSSKSLQNV |
| | | NGNTYLYWFQQKPGQSPQLLIYRMSNLNSGV |
| | | PDRFSGSGSGTEFTLTISSLEPEDFAVYYCMQ |
| | | HLEYPITFGAGTKLEIKRTVAAPSVFIFPPSDEQ |
| | | LKSGTASVVCLLNNFYPREAKVQWKVDNALQ |
| | | SGNSQESVTEQDSKDSTYSLSSTLTLSKADYE |
| | | KHKVYACEVTHQGLSSPVTKSFNRGEC |

### Working Examples

ADCC activity of MOR00208 and rituximab in DLBCL, MCL and CLL cell lines at various E:T ratios

### Example 1: Characterization of CD19 and CD20 Expression on Tested Cell Lines

### Methods & Data Analysis

In the present study, the QuantiBRITE™ system was used to quantify the amount of Phycoerythrin (PE)-labelled CD19 and CD20 antibodies bound per cell according to manufacturer's instructions. The QuantiBRITE™ system is based on four sets of beads coated with different pre-calibrated levels of PE-molecules, which were used to correlate Mean Fluorescence Intensity (MFI) values to the number of PE-molecules per bead. For each individual cell type, the measured MFI upon staining with PE-labelled antibodies was applied to a linear regression formula to calculate the respective Antibodies-bound-per-cell (ABC) values. The ABC values directly correlate with the number of CD19 and CD20 molecules per cell as the Biolegend CD19-PE (Biolegend #302208; clone HIB19) and CD20-PE (Biolegend #302306; clone 2H7) antibodies carry only one PE molecule per antibody. At a 1:1 labelling ratio of fluorochrome/ protein (F/P), MESF (Molecules of Equivalent Soluble Fluorochrome) values correspond to ABC values according to the equation MESF/ABC=effective F/P. Graph Pad PRISMTM software was used for the conversion of MFI into ABC values.

### Results

CD19 and CD20 expression levels on Toledo (DLBCL), MEC-1 (CLL) and JVM-2 (MCL) cells were analyzed. The QuantiBRITE™ system in combination with PE-labelled anti-CD19 and anti-CD20-antibodies was used to determine the expression levels of CD19 and CD20 on the tested B-cell tumor cell lines. For the DLBCL cell line Toledo, CD19 and CD20 expression levels of 35,721 and 28,008 antibodies bound per cell (ABCs) were determined (Table 1). CD19 and CD20 expression on MEC-1 cells (CLL) was quantified as 60,925 and 71,320 ABCs, whereas JVM-2 cells (MCL) showed CD19 expression levels of 26,157 and CD20 expression levels of 15,540 ABCs.

TABLE 1
CD19 and CD20 ABC values for JVM-2 (MCL),
Toledo (DLBCL) and MEC-1 (CLL) cells.

| Cell line | CD19 ABC | CD20 ABC |
|---|---|---|
| Toledo | 35,721 | 28,008 |
| MEC-1 | 60,925 | 71,320 |
| JVM-2 | 26,157 | 15,540 |

### Example 2: ADCC Activity Assays at Variable E:T Ratios

### Methods & Data Analysis

Antibody-dependent cell-mediated cytotoxicity (ADCC) is an immunologic cytotoxic effector mechanism, which is mainly dependent on the interaction of antibodies with Fc receptors on NK cells. ADCC is triggered when an antibody binds to a specific antigen on the surface of target cells, e.g. CD19 or CD20 on cancer cells, and the Fc fragment of the antibody interacts with Fc receptors on effector cells such as NK cells. This interaction activates the effector cells and the lysis of target cells is induced by the release of perforin and granzymes.

For the preparation of effector cells, peripheral blood mononuclear cells (PBMCs) were isolated from whole blood of healthy volunteers by density-gradient centrifugation with Biocoll separating solution and SepMate tubes. Next, NK cells were isolated from PBMCs via the MACS kit according to the manufacturer's protocol. Prior to incubation with antibodies and NK effector cells, one CLL cell line (MEC-1), one MCL (JVM-2) cell line and one DLBCL cell line (Toledo) were stained with 1 pM carboxyfluorescein succinimidyl ester (CFSE) for 3 minutes at room temperature.

In ADCC experiments, 2×10⁴ DLBCL, MCL or CLL target cells per well were incubated with NK cells as effector cells at varying effector to target (E:T) ratios and MOR00208 or Rituximab at a concentration of 10 μg/ml for 2 h at 37° C. and 5% CO₂. Unspecific NK cell mediated killing of tumor cells was determined by incubation of NK cells with target cells in absence of antibodies.

A flow cytometry based assay was utilized to measure the killing of target cells by quantifying dead and viable cells using 4′,6-diamidino-2-phenylindole (DAPI), a DNA intercalating dye, which is membrane impermeable and only intercalates in the DNA of dead cells with compromised membranes while being excluded from viable cells with intact membranes. Cells were stained with DAPI at a final concentration of 1 μg/ml and incubated for 10 minutes on ice, before the FACS measurement was performed.

Raw data were collected with a FACS Verse instrument and analysed with the FlowJo software. Cell populations were gated for living (DAPI negative) and dead target cells (DAPI positive). Data were exported to Microsoft Excel® to calculate the percentage of dead cells and percentage of specific killing with the following equations:

% dead cells=dead target cells/(dead target cells+viable target cells)×100 (sample)

% specific killing=% dead cells−% dead cells in NK and target cell control (w/o antibody).

Data analysis was performed with the statistical software package R and R Studio (Version 1.0.153 RStudio, Inc). For each independent experiment, % specific killing measured in triplicates was summarized with the geometric mean and its standard deviation. Furthermore, the ratio of specific killing was calculated from the % specific killing values by normalizing to the median value of Rituximab. For visualization, the geometric mean and its 95% confidence interval were calculated. The confidence interval of the geometric mean was calculated via bootstrap resampling with 1,000 iterations.

In order to summarize the data from all experiments per cell line, specific killing ratios of each donor were combined in a two-step process. First, the triplicate values from the individual experiments were aggregated to the geometric mean. Subsequently, the geometric mean values of the independent experiments were combined into one geometric mean value at the respective E:T ratio of an individual donor. At each E:T ratio, the median and its confidence interval were calculated via bootstrap resampling with 10,000 iterations based on the summarized geometric mean values of the individual donors (R Core Team 2017; Davison and Hinkley 1997; Wickham 2017).

### Results

ADCC activity of the Fc enhanced anti-CD19 antibody MOR00208 and the anti-CD20 antibody Rituximab towards Toledo (DLBCL), MEC-1 (CLL) and JVM-2 (MCL) cells was determined after a two-hour incubation with NK cells isolated from healthy human donors.

The anti-tumor activity of MOR00208 and Rituximab was evaluated at E:T ratios of 0.1:1, 0.3:1,1:1, 3:1 and 6:1 at an antibody concentration of 10 μg/ml. E:T ratios ranging from 0.1:1 to 6:1 were chosen as lower and upper ratio in the assay. The lowest ratio (1 NK cell vs 10 tumor cell; ratio 0.1:1) was determined by the minimal detectable ADCC signal in such an in vitro assay. The upper limit (6 NK cells vs 1 tumor cell; 6:1) was selected at a ratio where maximum of lysis is achieved under conditions of such in vitro assays.

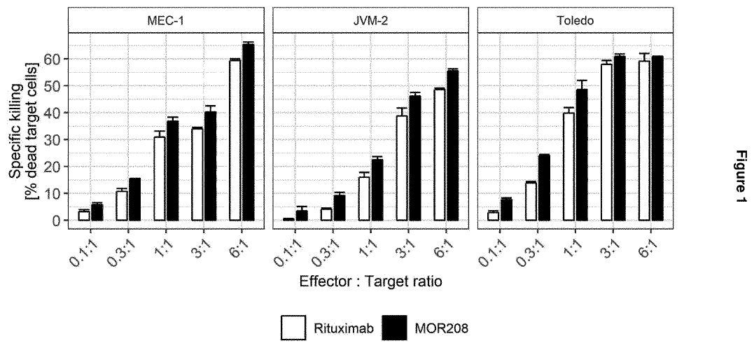

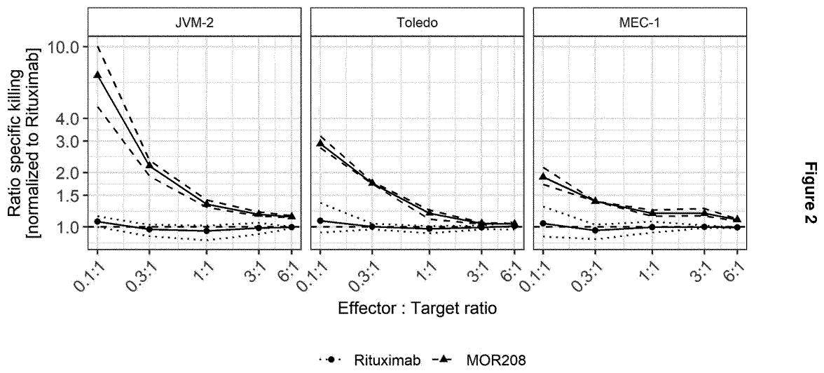

FIG. 1 shows representative results from an individual experiment for each target cell line depicted as % specific killing and FIG. 2 shows the ratio of specific killing MOR00208 normalized to Rituximab.

FIG. 1 shows one representative assay result of specific cell killing in MEC-1 cells mediated by MOR00208 (black) or Rituximab (white) in presence of NK cells in a two-hour assay at 37t. At the 3:1 and 6:1 E:T ratios, mean specific killing levels of 40 to 65% mediated by MOR00208 and 34-60% for Rituximab were found in MEC-1 cells. In the exemplary JVM-2 ADCC assay, specific killing of MOR00208 versus Rituximab was also elevated at the 3:1 and 6:1 E:T ratios in the range between 46 and 56% versus 39 and 48%. Toledo specific cell killing was similar for MOR00208 and Rituximab at the two higher E:T ratios with values of approximately 60%. Specific killing of Rituximab at the low E:T ratio of 0.1:1 was 3% of MEC-1 cells, whereas MOR00208 showed 6% specific killing. A similar finding was made in JVM-2 cells with an elevated specific cell killing of MOR00208 (3.4%) versus Rituximab (0.5%) and in Toledo cells with an increased cell killing of MOR00208 (8%) over Rituximab (3%) at the 0.1:1 E:T ratio.

The ratio of MOR00208 (black triangle) and Rituximab (circle) specific killing versus the median Rituximab value was 1.9 to 6.9 fold increased for all representative individual ADCC experiments with DLBCL, MCL and CLL cell lines at the 0.1:1 E:T ratios (FIG. 2). At higher E:T ratios of 3:1 and 6:1, specific killing reached a saturation level (see FIG. 1) and there was only a minimal difference in the ratio of specific killing of MOR00208 versus Rituximab (FIG. 2). All experimental data points were assessed in triplicates. In summary, E:T titrations were performed in ADCC assays with three B-cell tumor cell lines and NK cells of 61 blood samples isolated from 33 healthy blood donors. The ADCC activity of MOR00208 and Rituximab was evaluated with 8 donors for JVM-2 cells and 10 donors for Toledo cells in two independent experiments for each donor. MEC-1 cells were tested with 8 donors in two independent experiments and 9 further donors in single experiments.

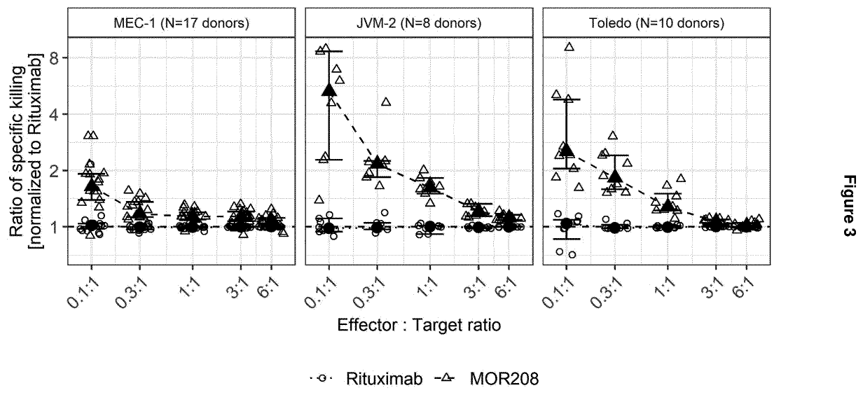

FIG. 3 shows the ratio of specific killing normalized to Rituximab for all experiments conducted with each cell line. Here, each circle or triangle represents the geometric mean value of two independent experiments performed in triplicates with NK cells from one individual blood donor. A specific killing ratio of 5.3 or 2.5 fold for MOR00208 normalized to Rituximab was found in JVM-2 or Toledo cells at an E:T ratio 0.1:1 depicted as median of numerous donors (FIG. 3). NK cells of individual donors showed an increased specific killing ratio of MOR00208 up to 20 or 30 fold compared to Rituximab at the lowest E:T ratio e.g. donor 296 with JVM-2 target cells or donor 299 with Toledo target cells (data not shown). In MEC-1 cells this effect was lower with an average of 1.6 fold increased specific killing ratio of MOR00208 over Rituximab at the 0.1:1 E:T ratio as result of multiple ADCC assays with NK cells isolated from 17 different donors. At high E:T ratios the increase of the specific killing ratio of MOR00208 compared to Rituximab was less distinct for all tested B-cell tumor cell lines, while at lower E:T ratios MOR00208 was clearly superior to Rituximab. It should be noted that the confidence intervals of MOR00208 as depicted in FIG. 3 did not overlap with the confidence intervals of Rituximab except for Toledo cells at an E:T ratio of 6:1 suggesting a general robustness regarding the observed superiority of MOR00208 vs. Rituximab. In conclusion, the ratio of specific killing increased towards lower E:T ratios and was most pronounced for JVM-2 and Toledo cells, while a consistent effect was visible for MEC-1 cells.

The monoclonal antibody MOR00208 targets the CD19 antigen on B cells and has two mutations (S239D and 1332E) in the Fc region to enhance antibody-dependent cell-mediated cytotoxicity. ADCC is a key mechanism for cancer cell killing, mainly mediated by tumor infiltrating NK cells. Bhat and Watzl 2007 showed increased serial killing of NK cells in presence of Rituximab with a maximal effect at low E:T ratios of 0.05:1, 0.1:1 and 0.2:1. Furthermore, it has been reported that Fc enhancement of antibodies results in increased serial killing of NK cells compared to the non-enhanced version of a CD33 specific antibody (Romain et al. 2014). Here, a 1.6 to 5.3 fold increased ratio of specific killing of MOR00208 normalized to Rituximab at a low E:T ratio of 0.1:1 with one cell line derived from DLBCL, one from MCL and one from CLL mediated by NK cells over a broad range of healthy donors has been demonstrated. The ratio of specific killing increased towards lower E:T ratios and was most pronounced for JVM-2 and Toledo, while a consistent trend was observed for MEC-1 cells. At higher E:T ratios of 3:1 and 6:1, MOR00208 mediated specific killing was similar to Rituximab at saturating antibody concentrations of 10 μg/ml.

Increased specific killing of MOR00208 compared to Rituximab at low E:T ratios was confirmed with freshly isolated NK cells from 33 healthy donors in 61 independent experiments in Toledo, JVM-2 and MEC-1 cells. These results show increased serial killing of NK cells at lower E:T ratios and provide evidence that MOR00208 has increased antitumor activity in DLBCL, MCL and CLL patients with low NK cell counts. In conclusion, the ADCC activity of MOR00208 showed most pronounced superiority relative to Rituximab under conditions where NK cells are limited. Therapies comprising the use of MOR00208 are therefore preferred over the standard of care (e.g. rituximab) for patients having a low NKCC at baseline.

### Example 3: T Cell and NK Cell Counting

As an example peripheral T and NK cell counting can be performed according to the following procedure:

T cells are a type of lymphocyte (a subtype of white blood cell) that play a central role in cell-mediated immunity. They can be distinguished from other lymphocytes, such as B cells and NK cells, by the presence of a T-cell receptor on the cell surface.

Natural killer cells or NK cells are a type of cytotoxic lymphocyte critical to the innate immune system. NK cells provide rapid responses to viral-infected cells, acting at around 3 days after infection, and respond to tumor formation. Typically, immune cells detect major histocompatibility complex (MHC) presented on infected cell surfaces, triggering cytokine release, causing lysis or apoptosis. NK cells are unique, however, as they have the ability to recognize stressed cells in the absence of antibodies and MHC, allowing for a much faster immune reaction.

### Materials and Methods

TriTest CD3 FITC/CD16+CD56 PE/CD45 PerCP (with TruCOUNT tubes), BD Biosciences, Cat: 340403 (US); 342442 (Europe). Pipettors and pipet tips capable of delivering 20 μL, 50 μL and 450 μL, Gilson Inc. FACS Lysing Solutions, BD Biosciences, Cat: 349202.

### Instruments: Flow cytometer, Vortex

### Background of Flow Cytometry

Whole blood is stained with fluorochrome-labeled antibodies (TriTEST) that bind specifically to leucocyte surface antigens. The cells travel past the laser beam and scatter the laser light. The stained cells fluoresce. These scatter and fluorescence signals, detected by the instrument, provide information about the cell's size, internal complexity, and relative fluorescence intensity. TriTEST reagents employ fluorescence triggering, allowing direct fluorescence gating of the NK- and T-cell lymphocyte population to reduce contamination of unlysed or nucleated red blood cells in the gate.

### Staining

For each patient sample, a TruCOUNT Tube is labelled with the sample identification number. 20 μL of TriTEST CD3/CD16+CD56/CD45 reagent was pipetted into the bottom of the tube. 50 μL of well-mixed, anticoagulated whole blood was pipetted into the bottom of the tube. Anticoagulated blood (EDTA) stored at room temperature (20-25° C.) must be stained within 24 hours of draw and analyzed within 6 hours of staining (keep at room temperature and protected from light). The tube is vortexed gently to mix. The tube is incubated for 15 minutes in the dark at room temperature (20-25° C.). 450 μL 1× FACS Lysing Solution is added to the tube. The tube is vortexed and incubated again for 15 minutes in the dark at room temperature (20-25° C.).

Using TruCOUNT Tubes, a known volume of sample is stained directly in a TruCOUNT Tube. The lyophilized pellet in the tube dissolves, releasing a known number of fluorescent beads. During analysis, the absolute number (cells/μL) of positive cells in the sample can be determined by comparing cellular events to bead events.

### Flow Cytometry

The cells are vortexed thoroughly (at low speed) to reduce aggregation before running them on the flow cytometer.

### Data Analyses

The CD45 vs SSC dot plot is visually inspected. Lymphocytes appear as a bright, compact cell population with low to moderate SSC. Monocytes (M) and granulocytes (G) appear as distinct populations. Analysis is completed when the cell populations of monocytes and lymphocytes showed clear separation.

Lymphocytes are first gated as CD45 positive, low SSC cell population. CD16/CD56 vs CD3 are pre-selected. T-cells (T) should appear as a compact bright CD3 positive cluster. NK-cells (NK) should appear as a compact bright CD16/CD56 positive cluster. Gating is completed and the T, and NK cells can be counted.

Bead event counts are done using a CD16/CD56 vs CD3 plot without any pre-selected gate. Beads should appear as a PE/FITC double positive cluster.

### Calculating Absolute Counts

The absolute number (cells/pL blood) of T cells or NK cells in the sample are determined by comparing cellular events to bead events. Either MultiSET software or manual (using CellQuest or other software) data analysis can be used. For manual counting, the number (#) of positive cellular acquired events is divided by the number (#) of acquired bead events, then multiplied by the (total TruCOUNT bead count (lot dependent) divided by whole blood sample volume of 50 μL). The result is absolute cell numbers per microliter.

### Equation

$$\frac{\begin{matrix}number of events in gate \\ containing cell population \\ ( T or NK )\end{matrix}}{\begin{matrix}number of events in gate 2 \\ containing bead population\end{matrix}} \times \frac{ of total TruCOUNT beads}{50 μ l whole blood} = cells / μ l blood$$

### Example

$$\frac{\begin{matrix}2709 acquired \\ T ‐ cells\end{matrix}}{10000 acquired beads} \times \frac{\begin{matrix}51667 total \\ beads in tube\end{matrix}}{50 μ l} = 280 T ‐ cells / μ l blood$$

We claim:

1. An anti-CD19 antibody for use in the treatment of hematological cancer patients wherein said patients have a peripheral NK cell count at baseline of less or equal to 100 NK cells/μl. use in

2. The anti-CD19 antibody for use in the treatment of hematological cancer patients according to claim 1, wherein the anti-CD19 antibody comprises an HCDR1 region comprising the sequence SYVMH (SEQ ID NO: 1), an HCDR2 region comprising the sequence NPYNDG (SEQ ID NO: 2), an HCDR3 region comprising the sequence GTYYYGTRVFDY (SEQ ID NO: 3), an LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6)

3. The anti-CD19 antibody for use in the treatment of hematological cancer patients according to any one of the preceding claims, wherein the anti-CD19 antibody comprises a variable heavy chain of the sequence
(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS
and a variable light chain of the sequence
(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

4. The anti-CD19 antibody for use in the treatment of hematological cancer patients according to any one of the preceding claims, wherein the anti-CD19 comprises a heavy chain having the sequence
(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTIPP
MLDSDGSFFLYSKLTVDKSRWQQGNVFSCSVMHEALHNHYTQKSLSLSPG
K
and a light chain having the sequence
(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

5. The anti-CD19 antibody for use in the treatment of hematological cancer patients according to any one of the preceding claims, wherein said anti-CD19 antibody is administered in combination with one or more additional pharmaceutical agent.

6. The anti-CD19 antibody for use in the treatment of hematological cancer patients according to claim 5 wherein said pharmaceutical agent is a biologic or a chemotherapeutic agent or a pharmaceutically acceptable salt thereof.

7. The anti-CD19 antibody for use in the treatment of hematological cancer patients according to claim 6, wherein said one or more pharmaceutical agent is a therapeutic antibody or antibody fragment, a nitrogen mustard, a purine analog, a thalidomide analog, a phosphoinositide 3-kinase inhibitor, a BCL-2 inhibitor, a bruton's tyrosine kinase (BTK) inhibitor or a pharmaceutically acceptable salt thereof.

8. The anti-CD19 antibody for use in the treatment of hematological cancer patients according to claim 7, wherein said one or more pharmaceutical agent is rituximab, R-CHOP, cyclophosphamide, chlorambucil, uramustine, ifosfamide, melphalan, bendamustine, mercaptopurine, azathioprine, thioguanine, fludarabine, thalidomide, lenalidomide, pomalidomide, idelalisib, duvelisib, copanlisib, ibrutinib, venetoclax or a pharmaceutically acceptable salt thereof.

9. The anti-CD19 antibody for use in the treatment of hematological cancer patients according to any of the preceding claims, wherein said hematologic cancer patient has chronic lymphocytic leukemia (CLL), non-Hodgkin's lymphoma (NHL), small lymphocytic lymphoma (SLL) or acute lymphoblastic leukemia (ALL).

10. The anti-CD19 antibody for use in the treatment of hematological cancer patients according to claim 9, wherein said hematologic cancer patient has non-Hodgkin's lymphoma.

11. The anti-CD19 antibody for use in the treatment of hematological cancer patients according to claim 10, wherein the non-Hodgkin's lymphoma is selected from the group consisting of follicular lymphoma, small lymphocytic lymphoma, mucosa-associated lymphoid tissue, marginal zone lymphoma, diffuse large B cell lymphoma, Burkitt's lymphoma and mantle cell lymphoma.

12. The anti-CD19 antibody for use in the treatment of hematological cancer patients according to claim 11, wherein the non-Hodgkin's lymphoma is Relapsed or Refractory Diffuse Large B-cell Lymphoma (r-r DLBCL).

13. The anti-CD19 antibody for use in the treatment of hematological cancer patients according to any of the preceding claims, wherein said anti-CD19 antibody increases one or more of the following features:
(i) the progression-free survival (PFS),
(ii) the objective response rate (ORR),
(iii) the duration of response (DoR),
(iv) the overall survival (OS),
(v) the time to progression (TTP).

14. A method of selecting a hematological cancer patient who is predicted to benefit from the therapeutic administration of an anti-CD19 antibody, said method comprising the following steps:
a) providing a blood sample obtained from said patient prior to treatment with said anti-CD19 antibody,
b) determining the peripheral NK cell count, and
c) selecting the patient on the basis of the patient has a peripheral NK cell count at baseline of less or equal to 100 cells/μl.

15. The method according to claim 14, further comprising the following step:
d) treatment of the selected patient with an anti-CD19 antibody.


SEQUENCE LISTING (13 sequences)

SEQ ID NO: 1 - protein, 5 aa, Artificial Sequence
  source: /note=“Description of Artificial Sequence Synthetic peptide”
SYVMH

SEQ ID NO: 2 - protein, 6 aa, Artificial Sequence
  source: /note=“Description of Artificial Sequence Synthetic peptide”
NPYNDG

SEQ ID NO: 3 - protein, 12 aa, Artificial Sequence
  source: /note=“Description of Artificial Sequence Synthetic peptide”
GTYYYGTRVFDY

SEQ ID NO: 4 - protein, 16 aa, Artificial Sequence
  source: /note=“Description of Artificial Sequence Synthetic peptide”
RSSKSLQNVNGNTYLY

SEQ ID NO: 5 - protein, 7 aa, Artificial Sequence
  source: /note=“Description of Artificial Sequence Synthetic peptide”
RMSNLNS

SEQ ID NO: 6 - protein, 9 aa, Artificial Sequence
  source: /note=“Description of Artificial Sequence Synthetic peptide”
MQHLEYPIT

SEQ ID NO: 7 - protein, 121 aa, Artificial Sequence
  source: /note=“Description of Artificial Sequence Synthetic polypeptide”
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 8 - protein, 112 aa, Artificial Sequence
  source: /note=“Description of Artificial Sequence Synthetic polypeptide”
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 9 - protein, 330 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 107 aa, Artificial Sequence [sequence omitted]

SEQ ID NO: 11 - protein, 451 aa, Artificial Sequence
  source: /note=“Description of Artificial Sequence Synthetic polypeptide”
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 219 aa, Artificial Sequence
  source: /note=“Description of Artificial Sequence Synthetic polypeptide”
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 556 aa, Homo sapiens [sequence omitted]